(19) United States
(12) Patent Application Publication
(10) Pub. No.: US 2005/0287169 A1
(43) Pub. Date: Dec. 29, 2005

(54) Methods of use of genes of pyridoxal 5'-phosphate biosynthesis in Bacillus subtilis: avirulent strains for vaccines, and methods for identification of antibacterial agents

(76) Inventor: Boris R. Belitsky, Swampscott, MA (US)

Correspondence Address: Sonia K. Guterman, Esq.;Lawson & Weitzen, LLP, Suite 345, 88 Black Falcon Avenue, Boston, MA, 02210-2414, US

(21) Appl. No.: 10/869,322

(22) Filed: Jun. 16, 2004

Related U.S. Application Data
(60) Provisional application No. 60/479,331, filed on Jun. 17, 2003.

(51) Int. Cl. A61K 39/02; C12N 15/74; C12N 1/21

(52) U.S. Cl. 424/200.1; 435/471; 435/252.3

(57) ABSTRACT

Methods and compositions comprising a pathogenic bacterial strain having a non-reverting mutation in a pdx gene encoding an enzyme involved in pyridoxal-5′-phosphate synthesis are provided, for use in vaccines, and methods for identification of inhibitors of the enzyme for use as an antibacterial agent are provided.

### RELATED APPLICATION

This application claims the benefit of U.S. provisional patent application having Ser. No. 60/479,331 filed in the U.S. Patent and Trademark Office on Jun. 17, 2003, and which is hereby incorporated by reference herein in its entirety.

### GOVERNMENT RIGHTS

A portion of this work was supported by a grant from the National Science Foundation (MCB-0110651). The government has certain rights in the invention.

### TECHNICAL FIELD

The invention relates to methods of making anti-bacterial vaccines, and methods of identifying novel anti-biological agents.

### BACKGROUND

Pyridoxal 5′-phosphate (PLP), the biologically active form of vitamin B₆, is an essential cofactor for numerous metabolic enzymes, such as aminotransferases, amino acid racemases, and amino acid decarboxylases, most of which have amino group-containing compounds as substrates [John, 1995]. In the absence of PLP, a substantial number of cellular biosynthetic and catabolic pathways would cease to function. PLP must be available to most bacteria even in rich media for production of D-amino acids, which are components of bacterial cell walls. Because only a few species of bacteria are capable of utilizing exogenous PLP, growth of most bacteria should depend on endogenous PLP synthesis.

Two pathways of de novo PLP synthesis are known. The PdxA/PdxJ pathway is present in some bacteria and the PDX1/PDX2 pathway can be found in Archaea, bacteria, fungi, sponges, plants, and plasmodia. Organisms appear to contain either one or the other pathway of de novo PLP synthesis, but none are known to contain both pathways. Vitamin B₆ comprises, in addition to PLP, precursors of PLP in phosphorylated and non-phosphorylated forms, and these compounds are referred to as B₆ vitamers. Non-phosphorylated vitamers pyridoxine, pyridoxal and pyridoxamine can be taken up by many bacteria, fungi, plants, and mammalian cells and converted into PLP by a salvage pathway.

A first de novo pathway, the PdxA/PdxJ pathway of PLP synthesis, comprises six dedicated steps, and has been extensively characterized in E. coli [Drewke, 2001; Hill, 1996]. A second pathway of PLP synthesis was recently discovered in fungi through identification of two proteins (SNZ/SOR1/PYROA/PDX1 and SNO/SNZB/PDX2) that are required for PLP synthesis [Osmani, 1999; Ehrenshaft, 1999; Ehrenshaft, 2001; Bean, 2001; Rodriguez-Navarro, 2002]. Genes encoding similar proteins are highly conserved in plants, sponges, plasmodia, Archaea, and many bacteria [Sivasubramaniam, 1995; Galperin, 1997; Mittenhuber, 2001; Seack, 2001], although a role for the proteins encoded by these genes in PLP synthesis or even functionality of encoded proteins in these organisms has never been established, and it is unclear whether steps of the PDX1/PDX2 pathway are common in different organisms.

The precise biochemical role of PDX1 is unknown; it may have a phosphate-binding site [Galperin, 1997] implicating a phosphorylated compound as a substrate. Another possible role for this site is binding of FMN (the IPR003009 motif at www.ebi.ac.uk/interpro). PDX2 is similar to some glutamine amidotransferases, consistent with the observation that in some fungi and bacteria the nitrogen group of PLP originates from glutamine; in E. coli and some other bacteria the nitrogen group of PLP originates from glutamate (42) through a SerC-catalyzed transamination step. PDX1 and PDX2 may function as an oligomeric complex (30).

No other components of the PDX1/PDX2 pathway are known. In yeast, a glucose-derived five-carbon-containing compound is utilized for PLP synthesis [Gupta, 2001]. Another possible PLP precursor in yeast was identified as 2′-hydroxypyridoxine or its phosphate derivative [Zeidler, 2002]. Roles for PDX1 and PDX2 orthologs in de novo synthesis of PLP have not been demonstrated for any bacterium.

### Summary of the Inventions

The invention in one embodiment provides a method of making an avirulent strain of a pathogenic bacterial species, which includes constructing a mutant cell of the species having a non-reverting mutation in a pdx gene encoding an enzyme involved in pyridoxal 5′-phosphate synthesis. The mutation for example, has a deletion. In another example, the mutation has an insertion. The mutation in the pdx gene is at least one from the group of pdxS, pdxT, and pdxZ mutations. In certain embodiments, the mutant cell has a pdx gene which is conditionally expressible. In some embodiments, the mutant cell has a pyridoxal growth requirement. In some examples, that growth requirement is not satisfied by pyridoxine. Further, growth of the mutant cell is substantially diminished in an infected subject compared to that of a cell of the pathogenic bacterial species.

In those embodiments, the bacterial species is from a genus selected from Actinobacillus, Bacillus, Campylobacter, Clostridium, Coxiella, Corynebacterium, Ehrlichia, Enterococcus, Francisella, Fusobacterium, Haemophilus, Helicobacter, Legionella, Leptospira, Listeria, Mannheimia, Mycobacterium, Neisseria, Neorickettsia, Pasteurella, Porphyromonas, Prevotella, Ralstonia, Staphylococcus, Streptococcus, Treponema, Tropheryma, Vibrio, Wigglesworthia, and Xylella. For example, the bacterial species is selected from the group of Actinobacillus pleuropneumoniae, Bacillus anthracis, B. cereus, Campylobacter jejeuni, Clostridium botulinum, Coxiella burnetti, Corynebacterium diphtheria, Enterococcus faecalis, Enterococcus faecium, Ehrlichia chaffeensis, Fusobacterium nucleatum, Francisella tularensis, Haemophilus ducreyi, Haemophilus influenzae, Helicobacter pylori, Legionella pneumophila, Leptospira interrogans, Listeria monocytogenes, Mannheimia haemolytica, Mycobacterium avium, M. bovis, M leprae, M tuberculosis, Neisseria meningitidis, Pasteurella multocida, Prevotella intermedia, Ralstonia solanacearum, Staphylococcus aureus, S. epidermidis, S. haemolyticus, Streptococcus agalactiae, S. mitis, S. pneumoniae, S. sobrinus, S. uberus, Treponema denticola, T. pallidum, Tropheryma whipplei, Vibrio cholerae, Wigglesworthia brevipalpis, and Xylella fastidiosa.

The invention in a related embodiment provides an avirulent strain produced according to the methods above. For example, the Clostridium avirulent strain produced is C. botulinum, the Bacillus is B. anthracis or B. cereus, the Campylobacter is C. jejuni, the Corynebacterium is C. diphtheriae, the Enterococcus is E. faecalis or E. faecium, the Francisella is F. tularensis, the Fusobacterium is Fusobacterium nucleatum, the Haemophilus is H. ducreyi or H. influenzae, the Leptospira is L. interrogans, the Listeria is L. monocytogenes, the Mycobacterium is selected from the group consisting of M avium, M bovis, M leprae, and M tuberculosis, the Staphylococcus is selected from the group consisting of S. aureus, S. epidermidis, S. haemolyticus, and the Streptococcus is selected from the group consisting of S. agalactiae, S. mitis, S. pneumoniae, S. sobrinus, S. uberus. A vaccine composition is provided that is prepared according to any of the methods above. The vaccine composition in related embodimens is provided in an effective dose. The vaccine composition in other related embodiments further comprises an adjuvant, or further comprises a pharmaceutically acceptable carrier, or both. A kit comprising a container and any of the above vaccines is also provided. The kit can also have instructions for use.

A method is provided of identifying from among a plurality of chemical compounds a potential antimicrobial agent that is inhibitory for synthesis of pyridoxal 5′-phosphate (PLP), the method comprising: contacting a first sample of cells of a bacterial test strain with at least one of the plurality of compounds, wherein the strain has a pathway of de novo PLP synthesis; and comparing growth of the first sample with that of a second sample not so contacted and otherwise identical, and with a third sample similarly contacted and in the presence of excess vitamin B₆, such that inhibition of growth of cells in the first sample in comparison to growth of cells in the second and third samples is an indication that the compound is an agent inhibitory of PLP synthesis. In one embodiment, the de novo pathway of synthesis in the test strain is a PdxST pathway. In a related embodiment, the test strain is selected from the group consisting of: Bacillus subtilis, Geobacillus stearothermophilus, and Listeria monocytogenes. The method in related embodiments further comprises contacting a sample of cells of a control strain having a non-PdxST de novo PLP pathway for PLP synthesis with the at least one compound, wherein lack of inhibition of growth of cells of the second strain is a further indication that the compound is an inhibitor of the PdxST pathway. The non-PdxST de novo pathway is a PdxAJ pathway. The control strain is an E. coli. The test strain is selected from the group consisting of: Actinobacillus pleuropneumoniae, Bacillus anthracis, B. cereus, Campylobacter jejeuni, Clostridium botulinum, Corynebacterium diphtheria, Coxiella burnetti, Ehrlichia chaffeensis, Enterococcus faecalis, E. faecium, Fusobacterium nucleatum, Francisella tularensis, Haemophilus ducreyi, Haemophilus influenzae, Helicobacter pylori, Legionella pneumophila, Leptospira interrogans, Listeria monocytogenes, Mannheimia haemolytica, Mycobacterium avium, Mycobacterium bovis, Mycobacterium leprae, M tuberculosis, Neisseria gonorrhoeae, N. meningitidis, Neorickettsia sennettsu, Pasteurella multocida, Prevotella intermedia, Porphyromonas gingivalis, Ralstonia solanacearum, Staphylococcus aureus, S. epidermidis, S. haemolyticus, Streptococcus agalactiae, S. mitis, S. pneumoniae, S. sobrinus, S. uberus, Treponema denticola, T. pallidum, Tropheryma whipplei, Vibrio cholerae, Wigglesworthia brevipalpis, and Xylella fastidiosa.

Also provided is a method of identifying from among a plurality of chemical compounds a potential antimicrobial agent that is inhibitory for synthesis of pyridoxal 5′-phosphate (PLP), the method comprising: contacting a first sample of cells of a bacterial strain grown in the presence of vitamin B₆ with at least one of the plurality of compounds, wherein the strain has a PLP salvage pathway having a PdxZ pyridoxal kinase and mutationally lacking a de novo PLP synthesis pathway; and comparing growth of the first sample with a second sample of cells not contacted with the compound and otherwise identical and with a third sample of the bacterial strain otherwise identical and in the presence of an effective amount of a B6 vitamer, wherein inhibition of growth of the first sample compared to the second sample and the third sample indicates that the compound is an inhibitor for synthesis of PLP.

In the method, the effective amount for growth of the third sample of cells is at least 1 mM of the vitamin B₆ vitamer. For example, the effective amount for growth of the third sample of cells is at least at least about 2 mM of the vitamin B₆ vitamer. The bacterial species is a genus selected from Actinobacillus, Bacillus, Campylobacter, Clostridium, Coxiella, Corynebacterium, Ehrlichia, Enterococcus, Francisella, Fusobacterium, Haemophilus, Helicobacter, Legionella, Leptospira, Listeria, Mannheimia, Mycobacterium, Neisseria, Neorickettsia, Pasteurella, Porphyromonas, Prevotella, Ralstonia, Staphylococcus, Streptococcus, Treponema, Tropheryma, Vibrio, Wigglesworthia, and Xylella.

Also provided is a method of identifying from among a plurality of chemical compounds a potential antimicrobial agent that is inhibitory for synthesis of pyridoxal 5′-phosphate (PLP), the method comprising contacting a first sample of a bacterial PdxS-PdxT enzyme complex with at least one of the plurality of compounds; and comparing enzymatic activity of the first sample with that of a second sample not so contacted and otherwise identical, wherein inhibition of activity in the first sample in comparison to activity in the second samples is an indication that the compound is an agent inhibitor of PLP synthesis. The method can in a related embodiment comprise a third and a fourth sample having an enzyme that is not PdxS-PdxT, the third sample being contacted with the compound and the fourth sample not so contacted and otherwise identical to the third, wherein absence of inhibition of the third sample in comparison to the fourth sample is a further indication that the compound is a specific inhibitor of PdxS-PdxT. For example, the enzyme in the third and fourth samples is β-galactosidase, alkaline phosphatase, α-amylase, or horse radish peroxidase. In general, comparing enzymatic activity is measuring a glutaminase activity. The bacterial PdxS-PdxT enzyme is obtained from a strain selected from the group a B. subtilis, a G. stearothermophilus, and a Listeria monocytogenes. The bacterial PdxS-PdxT enzyme is obtained from a strain selected from a bacterial strain from an Actinobacillus, a Bacillus, a Campylobacter, a Clostridium, a Coxiella, a Corynebacterium, an Ehrlichia, an Enterococcus, a Francisella, a Fusobacterium, a Haemophilus, a Helicobacter, a Legionella, a Leptospira, a Listeria, a Mannheimia, a Mycobacterium, a Neisseria, a Neorickettsia, a Pasteurella, a Porphyromonas, a Prevotella, a Ralstonia, a Staphylococcus, and a Streptococcus, a Treponema, a Tropheryma, a Vibrio, a Wigglesworthia, or a Xylella. The enzyme can be isolated. The enzyme can further have a modification. For example, at least one of PdxS or PdxT has additional histidine residues.

### BRIEF DESCRIPTION OF THE DRAWINGS

FIG. 1 is a sketch of pathways of PLP synthesis in which de novo PLP synthetic pathways from common cellular metabolites are shown from top to bottom, and salvage synthesis pathways for vitamers pyridoxine, pyridoxal and pyridoxamine are shown from right to left.

FIG. 2 is a drawing that shows a genetic map of Bacillus subtilis pdxST (yaaDE) region and plasmids carrying different parts of this region. The restriction sites are abbreviated as follows: A, AatII; B, BamHI; Bs, BseRI; E, EcoRI; H, HindIII; M, MfeI; N, EcoNI; S, SalI; Su, Sau3A; Sp, SphI; X, XbaI. Only relevant EcoNI, MfeI, and Sau3A sites are indicated. The sites shown in parentheses were created by PCR. Deletion mutations are shown by triangles. The 1.4-kb ble cassette is not drawn to scale. An asterisk shows location of a missense mutation at the end of the pdxT gene obtained during the cloning manipulations.

FIG. 3A is a line graph showing growth of B. subtilis ΔpdxS with different concentrations of pyridoxal (PL). Growth rate is a function of PL concentration, between a minimal concentration (no growth observed at 0.01 μM) of 0.03 μM and 10 μM.

FIG. 3B is a line graph showing growth of the pdxT (yaaE) mutant in medium supplemented with different ammonium salt concentrations. Cells were grown overnight in TSS-glucose medium with 30 mM ammonium, and were diluted 100-fold into fresh medium with the indicated ammonium concentrations supplied as ammonium chloride.

FIG. 4 is a photograph of an SDS-PAGE electrophoretogram showing purification of PdxS and PdxT proteins, and the PdxST protein complex. Lane 1: Pdx S; Lane 2, PdxST complex comprising wild type PdxS and PdxT-H6; Lane 3, PdxT.

FIG. 5 is a line graph that shows glutaminase activity measured for each of PdxT protein (triangles) and a complex of PdxS and PdxT (circles) as a function of time (minutes) on the abscissa.

FIG. 6 is a line graph showing expression of a pdxS-lacZ fusion during growth in rich medium (LB broth).

### DETAILED DESCRIPTION OF EMBODIMENTS

Cells of the bacterium Bacillus subtilis are herein envisioned to utilize the PDX1/PDX2 pathway of PLP biosynthesis, as nucleic acid sequences similar to genes of this pathway are present in the B. subtilis genome [Kunst, 1997] (http://genolist.pasteur.fr/SubtiList) located adjacent on the chromosome. These genes, previously identified only as genes of unknown function having names yaaD and yaaE, were discovered herein as homologous to PDX1 and PDX2, respectively.

Uncharacterized B. subtilis mutants requiring pyridoxine, pyridoxal, or pyridoxamine for growth have been described [Pflug, 1978]. These mutations were not further characterized. However, these cells have the ability to utilize the unphosphorylated vitamers for growth. It was not known whether these B. subtilis mutants might have PLP salvage reactions. A kinase activity but not oxidase activity of the E. coli-like salvage pathway was detected in B. subtilis cell extracts [Pflug, 1978][Pflug, 1983]. However, no information on PLP metabolism in other bacteria containing the PDX1/PDX2 pathway has been available.

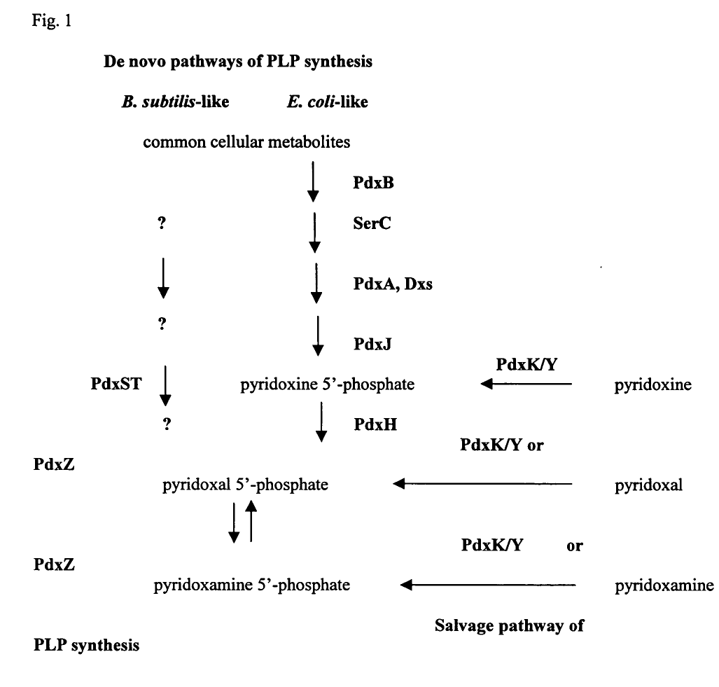

In the salvage pathway, simple B₆ vitamers pyridoxine, pyridoxal and pyridoxamine are phosphorylated by pyridoxal kinase (PdxK or PdxY in E. coli) to provide pyridoxine 5′-phosphate, PLP, and pyridoxamine 5′-phosphate, respectively. The latter two compounds can be used as cofactors in enzymatic reactions, however pyridoxine must be oxidized by pyridoxine 5′-phosphate oxidase, PdxH (see FIG. 1). Genes encoding pyridoxal kinase-like enzymes are absent from the genomes of many bacteria [Mittenhuber, 2001], indicating that many species of bacteria have a salvage pathway different from that found in E. coli and mammals.

Examples herein show that YaaD (renamed herein as PdxS) and YaaE (renamed as PdxT) form a glutamine amidotransferase required for a single de novo pathway of PLP synthesis in Gram positive bacteria, including B. subtilis, Geobacillus stearothermophilus, and Listeria monocytogenes.

Vitamin B₆ biosynthesis in B. subtilis has not been characterized previous to this work. Null mutations in two genes that might be involved in B₆ biosynthesis, based on the comparative genomic analysis, were constructed. The pdxS (yaaD) mutant thus constructed was found herein to be a strict B₆ auxotroph, and the pdxT (yaaE) mutant was found to be a conditional auxotroph, the phenotype depending on availability of ammonium in growth medium. Pyridoxal was the predominant B₆ vitamer utilized by B. subtilis cells. The adjacentpdxS andpdxT genes form an operon expression of which is not dependent on B₆ availability in any straightforward relationship. PdxS and PdxT copurify during affinity chromatography, and apparently the two proteins form a complex. The mixture of purified PdxS and PdxT has glutaminase activity. PdxT is similar to glutaminase subunits of some glutamine amidotransferases. It is shown herein that PdxS and PdxT encode the synthase and glutaminase subunits, respectively, of a glutamine amidotransferase that is essential for B₆ biosynthesis in B. subtilis, and is essential in many bacteria, Archaea, fungi, plants, and other organisms.

D-amino acids are not present in animal cells in any significant amounts, hence pathways involved in their synthesis are reasonable targets for identification of antibacterial compounds. Metabolites such as diaminopimelic acid, that are synthesized by PLP-dependent enzymes and that are required for bacterial growth may also be limiting during infection. Thus, even partial PLP limitation may cause severe negative effects on bacterial growth and thereby reduce virulence of many pathogenic bacteria. Inhibition of bacterial PLP synthesis could be an important goal for antibacterial chemotherapy. Naturally occurring vitamin B₆ antimetabolites have been identified, for example the plant compound ginkgotoxin. The toxicity of ginkgotoxin (4′-O-methylpyridoxine), which acts in vivo as a neurotoxin, is alleviated by vitamin B6 [Buss, 2001]. Thus interference with PLP biosynthesis provides a potentially efficacious target for development of antimicrobial agents.

No published information is available on a bacterial requirement for PLP synthesis during growth in bacterially-infected subject animals. The sum of amounts of exogenous and/or endogenous B₆ vitamers is tissue-dependent, and B6 availability is likely to be different for extracellular or intracellular bacteria and for bacteria replicating in the cytosol or inside vacuolar compartments. Therefore, the severity of the effect of PLP limitation on growth in the host may depend on the niche the bacteria occupy in the infected subject. As it is possible that the two types of pathways of PLP synthesis, de novo and salvage, have different relative roles during bacterial growth and at various stages of infection, an integrative approach, focusing on both pathways of PLP synthesis, would be more successful to screen for therapeutic agents than use of each pathway separately, because PLP is essential for growth of most bacteria in infected host animals.

Mammalian cells are not able to synthesize PLP de novo. In bacteria, some proteins of de novo PLP pathways, including PDX1 and PDX2, are dissimilar to mammalian proteins, or have only very limited similarity. Therefore, PDX1- and PDX2-like proteins are potential targets for antibacterial drugs.

Since plasmodia, fungi, and plants as well as bacterial species possess the PDX1/PDX2 pathway of de novo PLP synthesis, potential therapeutic agents obtained from methods of targeting this pathway of PLP synthesis can have broad range anti-biological activities, for example, can also have antimalarial, antifungal, and herbicidal as well as antibacterial activities. Novel antibacterial antibiotics are increasingly needed as many common pathogenic bacteria acquire resistance to known antibiotics.

Mammalian PLP salvage enzymes that are kinases and oxidases share a significant degree of similarity with these enzymes in E. coli and several other bacterial species. Drugs directed against these particular bacterial enzymes might interfere with PLP synthesis by host organism subject cells, as well as by infecting microbes. However, in the genomes of many bacteria, including pathogenic bacteria no gene that might encode a product substantially similar to mammalian enzymes is present. Therefore, if novel enzymes of the PLP salvage pathway are idenified, they could serve as potential drug targets. The gene encoding salvage enzyme pyridoxal kinase is shown herein to be present in B. subtilis (originally designated as a gene of unknown function ywdB, also identified as thiD), and is here renamed pdxZ.

In addition, mutants of pathogenic bacteria which are defective in PLP pathways can be less virulent (i.e., are attenuated or avirulent) in animal subjects including humans, so that these mutant strains could also serve as potential vehicles for vaccine strain development. Therefore, construction of PLP defective bacterial mutants is a novel approach for providing attenuated bacteria which can be used as vaccine strains.

The bacterial strains described herein, recombinant bacterial strains which are the recombinantly-produced pdx mutants and equivalents of the present invention, can be tested in animal subjects or on plant crop subjects in vivo, and efficacy at high and low doses can be established. An “effective dose” of a vaccine or an antibacterial agent is an amount of the composition that prevents and/or remediates either or both of a clinical symptom associated with a bacterial infection, and decreases or eliminates death of members of a subject population due to the bacterial infection. Animal subjects include mammals such as rodents, apes, carnivores, farm animals, agricultural animals, and insects. Rodents are exemplified by mice, rats, rabbits, and guinea pigs. Mammals as defined herein to include human subjects, both without clinical symptoms and also symptomatic patients.

Further in vivo use of these and other strains provided herein can be tested for safety in animals. In this case, the strains may be administered to the animal orally or directly into the stomach. The animals may be sacrificed a few days (for example, 1 to 3 days) after the administration of the bacteria.

The attenuated bacterial strains, such as recombinant bacterial strains, such as pdx mutants may also be administered to the animal, e.g., by intraperitoneal injection. Organs of sacrificed animals such as spleen and liver can be examined for the presence of intracellular bacteria, an indication of insufficient safety. Intracellular bacteria may be detected by for example, cultivating cell extracts on solid medium. A strain may be employed in an immunogenic composition to induce an immune response for treating various pathological conditions in mammals. The pathological conditions contemplated by the present invention include pathogen infections as disclosed herein. The immunogenic compositions can include, in addition to bacteria, other substances such as cytokines, adjuvants and pharmaceutically acceptable carriers. Cytokines can also be included in such immunogenic compositions using, for example, additional recombinant bacteria of the present invention capable of delivering a cytokine. These immunogenic compositions may be administered to the subject in any convenient manner, such as orally, intraperitoneally, intravenously or subcutaneously. Specific immune responses induced by such compositions can lead to CTL-mediated or antibody-mediated killing of the pathogens or cells with abnormal expression of a relevant antigen, thus alleviating the relevant pathological condition.

The present invention is further illustrated by the following examples, which are exemplary only and are not meant to be further limiting. All the publications mentioned in the present disclosure are incorporated herein by reference in their entirety. The terms and expressions which have been employed in the present disclosure are used as terms of description and not of limitation, and there is no intention in the use of such terms and expressions of excluding any equivalents of the features shown and described or portions thereof, it being recognized that various modifications are possible within the scope of the invention.

### EXAMPLES

The following Materials and Methods were used throughout the Examples below.

Bacterial strains, plasmids and culture media. B. subtilis strains used in this study are listed in Table 1. B. subtilis strains were grown at 37° C. in TSS minimal medium with 0.5% glucose as the carbon source and 0.2% glutamate or NH₄Cl as nitrogen source, in LB broth [Miller, 1972], or in DS nutrient broth medium [Fouet, 1990]. The same media with addition of agar were used for growth of bacteria on plates. LB broth or LB agar was used for growth of Escherichia coli strains. The following antibiotic concentrations were used as appropriate: chloramphenicol, 2.5 μg/ml; neomycin, 2.5-5 μg/ml; phleomycin, 0.25 μg/ml; spectinomycin, 50 μg/ml; or a combination of erythromycin, 0.5 μg/ml, and lincomycin, 12.5 μg/ml, for B. subtilis strains; ampicillin, 50-100 μg/ml; kanamycin, 25 μg/ml; spectinomycin, 50 μg/ml; or chloramphenicol, 10 μg/ml, for E. coli strains.

Plasmids used in this work are described below and shown in FIG. 2. E. coli strain JM107 [Yanisch-Perron, 1985] was used for propagation of most plasmids.

DNA manipulations and transformation. Methods for plasmid isolation, agarose gel electrophoresis, use of restriction and DNA-modification enzymes, DNA ligation, PCR, and electroporation of E. coli cells were as described by Sambrook et al. [Sambrook, 1989]. Isolation of chromosomal DNA and transformation of B. subtilis cells by chromosomal or plasmid DNA were as described [Belitsky, 1998]. Platinum High Fidelity Taq or Pfx polymerases (Invitrogen) were used to generate fragments for cloning; all cloned PCR-generated fragments were verified by sequencing.

Cloning of the yaaDE region. A 1.8-kb PCR product containing the yaaD (pdxS) and the yaaE (pdxT) genes was synthesized using SMY chromosomal DNA as template and yaaDE-specific primers, oBB120 (5′-GATTTGGATCCGGAATTTGGGGAAGC; SEQ ID NO:1) and oBB121 (5′-TTATAGTCGACTATAAGTTTCCACAGC; SEQ ID NO:2), containing BamHI and SalI sites (in bold). The PCR fragment was cloned in pBB544, an integrative vector conferring neomycin resistance [Belitsky, 1997]. The resulting plasmid, pBB1181 (FIG. 2), contained a point mutation in the penultimate codon of the yaaE gene (GGC to AGC with a corresponding Gly to Ser change) and was stable only in the pcnB80 zad::Tn10 derivative of E. coli strain JM107 which reduces the plasmid copy number (Lopilato, 1986,) and hence was observed to alleviate apparent toxicity of the cloned fragment.

Construction of yaaD and yaaE null mutants. A deletion mutation within gene yaaD was created by cutting out the 606-bp fragment starting at the AatII site of pBB1181 (FIG. 2) and religating the remaining fragment after filling in its ends (the deletion endpoint, as determined by sequencing, was determined to be beyond the HindIII site, designed for this construct). In the resulting plasmid, pBB1185 (FIG. 2), the deletion was found not to alter the reading frame ofyaaD. pBB1218, a truncated version of pBB1185 (FIG. 2), was introduced by a single-crossover, homologous recombination event into the chromosome of strain SMY, selecting transformants for neomycin resistance. About 25% of these transformants, merodiploid for the yaaD region, acquired the Pdx⁻ phenotype (small colony phenotype on DS plates and inability to grow in minimal medium in the absence of B6 vitamers), indicating that this phenotype is conferred by the yaaD deletion and that this region in recipient calls most probably was subject to gene conversion of the cellular genome to a homozygous ΔyaaD/ΔyaaD state (the Pdx⁻ phenotype conferred by the yaaD deletion was found to be recessive). After growth of cells of the Neo^{S} Pdx⁻ strain in the absence of neomycin, a Neos Pdx⁻ derivative was isolated. In this strain it was observed that the integrated pBB1218 had been excised from the chromosome and lost. Replacement of the wild-type yaaD gene by the ΔyaaD allele in strain BB2253 and excision of pBB1218 was confirmed by sizing PCR fragments generated from the yaaD locus and by probing for the loss of vector sequences.

A deletion mutation covering a 3′-part of the yaaD gene and a 5′-part of the yaaE gene was constructed in pBB1190 (FIG. 2) by cutting out the 898-bp fragment starting at the Aatll site of pBB1181 and religating the remaining fragment after filling in its ends. To construct strain BB2254 (ΔyaaDE), this mutation was introduced into the chromosome of strain SMY as described above for the ΔyaaD mutation.

A deletion-insertion mutation within the yaaE gene was constructed by replacing a 135-bp SphI-EcoRI fragment of pBB1213 (FIG. 2) with a 1.4-kb SphI-EcoRI ble cassette determining resistance to phleomycin, excised from pJPM136 [Belitsky, 1998]. The orientation of the ble gene in the resulting plasmid pBB1217 (ΔyaaE::ble; FIG. 2) coincided with that of the yaaE gene. Plasmid pBB1217 was introduced into B. subtilis SMY, and Phlr NeoS transformants arising from double crossover homologous recombination events were selected. Replacement of the chromosomal yaaE gene by the ΔyaaE::ble allele in strain BB2256 was confirmed by comparing sizes of PCR fragments from each of the wild-type and the mutant yaaE chromosomal loci.

Construction of transcriptional pdxS-lacZ (yaaD-lacZ) and pdxT-lacZ (yaaE-lacZ) fusions. The 1.35-kb BamHI-EcoRI fragment containing the entire intragenic region between dacA and pdxS, the entire pdxS gene, and the 5′-end of the pdxT gene (FIG. 2), was cut out from the PCR product generated using primers oBB120 and oBB121 and was cloned between the corresponding sites of pHK23 to construct pBB1254 (pdxST-lacZ; FIG. 2). Plasmid pHK23 containing an erythromycin resistance gene was derived from pLG103 [Belitsky et al., 2002, the entire contents of which are hereby incorporated by reference herein]. To construct plasmid pBB1259 with apdxS-lacZ fusion (FIG. 2), a 0.50-kb HindIII-EcoRI fragment containing the 3′-end of the pdxS gene and the 5′-end of thepdxT gene, was cut out of pBB1254, and the plasmid was blunt-ended and religated. To construct plasmid pBB1262 with a pdxT-lacZ fusion (FIG. 2), a 0.43-kb BamHI-BseRI fragment, containing the pdxS promoter and the 5′-part of the pdxS gene, was cut out of pBB1254, and the plasmid was blunt-ended and religated. Strains BB2277, 2279, and 2285 carrying thepdx-lacZ transcriptional fusions integrated at the amyE locus were isolated after transforming strain SMY with pBB1254, 1259, and 1262, respectively, selecting for resistance to erythromycin and screening for loss both of α-amylase production (Amy) and vector sequences as indications of a double crossover, homologous recombination event. The Amy phenotype was assayed using colonies grown overnight on Tryptose Blood Agar Base (Difco)-0.2% starch plates [Fouet, 1990].

Each of plasmids pBB1254 and pBB1259 was also integrated by a single crossover recombination event at the pdxST locus of the SMY chromosome, to construct strains BB2278 and BB2280, respectively. Location of the fusions in these strains was confirmed by genetic mapping in transformation experiments.

Construction of a pdxS-lacZ translational fusion. To construct a translational pdxS-lacZ fusion in plasmid pBB1263 (FIG. 2), a 0.58-kb MfeI-Sau3A fragment containing all of the intergenic region between dacA and pdxS, and the 5′-part ofpdxs was cut out from one of the pdxST PCR products, and was inserted between the EcoRI and BamHI sites of pJPM96 [Belitsky, 1997]. The constructed fusion was integrated by a single crossover recombination event at the pdxS locus of the SMY chromosome to create strain BB2294.

PdxST overexpression. A 1.54-kb DNA fragment coding for all of a wild-type version of PdxS and a modified version of PdxT containing a His₆-tag at its C-terminus was synthesized by PCR, using SMY chromosomal DNA as a template, and oligonucleotides oBB138 (5′-ATAACGGATCCTTGATTAGGGGGACC; SEQ ID NO:3), and oBB144 (5′TTTCAGTCGACTTAATGGTGATGGTGATGGTGTACAAGTGCCTTTTGCTTAT; SEQ ID NO:4), as 5′- and 3′-primers, respectively (BamHI and SalI sites are in bold). The PCR fragment was first blunt-ended, then cut with SalI, and was then cloned into expression vector pBAD 18 containing the inducible E. coli ara promoter [Guzman, 1995] and was then cut with SmaI and SalI. Two derivatives of the resulting plasmid, pBB1252 (FIG. 2), expressing either PdxT-His₆ only (pBB1256) or unmodified PdxS only (pBB1257) were generated by cutting out either the 0.37-kb Acc65I (the vector site)-SwaI fragment containing the 5′-part of the pdxS gene or the 0.54-kb fragment containing most of the pdxT gene between the insert and vector SphI sites (FIG. 2).

Plasmid pBB1261 (FIG. 2) overexpressing the PdxS-His₆ protein was constructed by cloning the 0.90-kb NcoI-SphI fragment in the pBAD24 vector containing the E. coli ara promoter and an appropriate ribosomal binding site [Guzman, 1995]. The pdxS fragment was synthesized by PCR using SMY chromosomal DNA as a template, and oligonucleotides oBB151 (5′-CCAAGCCATGGCTCAAACAGGTAC; SEQ ID NO:5), and oBB152 (5′TGTTCGCATGCTTAATGGTGATGGTGATGGTGCCAGCCGCGTTCTTGCAT; SEQ ID NO:6), as 5′- and 3′-primers, respectively (NcoI and SphI sites are indicated in bold).

pBAD derivatives were introduced into E. coli LMG194 (ara⁻) [Guzman, 1995] cells by transformation. The PdxS and/or PdxT proteins were induced in L-broth cultures (A₆₀₀=0.25−0.4) by addition of 0.2% L-arabinose and incubation for four hours. Cells were pelleted, were washed in 50 mM tris-Cl (pH 8.0)-5% glycerol and were disrupted by sonication in modified Novagen binding buffer (20 mM tris-Cl (pH 7.9)-500 mM NaCl-5 mM imidazole-5% glycerol) plus 1 mM phenylmethylsulfonyl fluoride and 0.1% Nonidet P-40. The sonicate was clarified by low speed centrifugation, and the supernatant was purified using a 2 ml Ni²⁺-column (Novagen) as described by the manufacturer. Buffers containing increasing concentrations of imidazole, from 30 mM to 1 M, were initially used for elution, and 485 mM and 385 mM imidazole were found suitable for elution of PdxS and PdxT proteins, respectively.

Cloning of the B. subtilis pdxz region. A 1.4-kb PCR product containing the pdxZ (ywdB, thiD) gene and its flanking sequences was synthesized using SMY chromosomal DNA as template andpdxz-specific primers, oBB1182 (5′-CGTTCAAGCTTGTGAAGAGAAGG; SEQ ID NO: 7) and oBB123 (5′-AAGCGTCTAGAATATAAGGATGTGC; SEQ ID NO: 8), containing HindIII and XbaI sites (shown in bold). The PCR fragment was cloned into plasmid pBB544, an integrative vector conferring neomycin resistance [Belitsky, 1997], to construct plasmid, pBB1182.

Construction of a pdxZ null mutant. A deletion-insertion mutation within the pdxZ gene was constructed by replacing the 0.32-kb NheI-EcoRI fragment of pBB1182 with a 1.1-kb SpeI-EcoRI spc cassette conferring resistance to spectinomycin, the cassette originally obtained from pJL73 [LeDeaux, 1995]. The orientation of the spc gene in the resulting plasmid pBB1184 (ΔpdxZ::spc) was that of the pdxZ gene. Plasmid pBB1184 was introduced into B. subtilis SMY and Spc^{r} Neo^{S} transformants, arising from double crossover homologous recombination events, were selected. Replacement of the chromosomal pdxZ gene by the ΔpdxZ::spc allele in strain BB2251 was confirmed by comparing the sizes of PCR fragments synthesized using the wild-type and mutant pdxZ chromosomal loci as templates.

DNA sequencing. DNA fragments from the pdxST region were sequenced using vector- or pdx-specific oligonucleotides as primers (by the Tufts University Core Facility) or by the dideoxy chain termination method [Sanger, 1977] using the Sequenase reagent kit (Amersham) as recommended by the manufacturer. Plasmid double-stranded DNA to be sequenced was purified using a QIAprep Spin Miniprep Kit (Qiagen). DNA and protein sequences were analyzed using the DNA Strider and BLAST programs [Altschul, 1997; Marck, 1988].

Enzyme assays. To determine glutaminase activity, samples of purified PdxT or (and) PdxS were assayed at room temperature in 1 ml of a buffer containing 50 mM Tris-Cl (pH 8.2), 5 mM NaCl, 5 mM imidazole, 0.5% glycerol, 10 mM glutamine, 0.6 mM 3-acetylpyridine-NAD (APAD), and 1 unit of bovine glutamate dehydrogenase (Sigma). Reduction of APAD by glutamate dehydrogenase coupled to conversion of the product of glutaminase reaction, glutamate, to 2-ketoglutarate was monitored as an increase in absorption at 363 nm; very low non-specific reduction of APAD was observed in reactions lacking glutamine. One unit of enzyme activity was defined as the amount needed to convert 1 nmol of APAD to APADH (per min per mg of protein). Protein concentration was determined using the Bio-Rad Protein Assay (Bio-Rad) with bovine serum albumin as a standard.

Activity of β-galactosidase was determined as described previously [Belitsky, 1995]. Specific activities are expressed in Miller units (1000×OD₄₂₀/min·ml·OD₆₀₀) [Miller, 1972].

### Example 1

### Isolation of yaaD and yaaE Null Mutants

An in-frame deletion mutation in the yaaD gene was designed so as not to affect expression of the downstream yaaE gene, and this strain and a deletion-insertion mutation in the yaaE gene were each constructed, and were separately introduced into the B. subtilis chromosome as described in Materials and Methods herein.

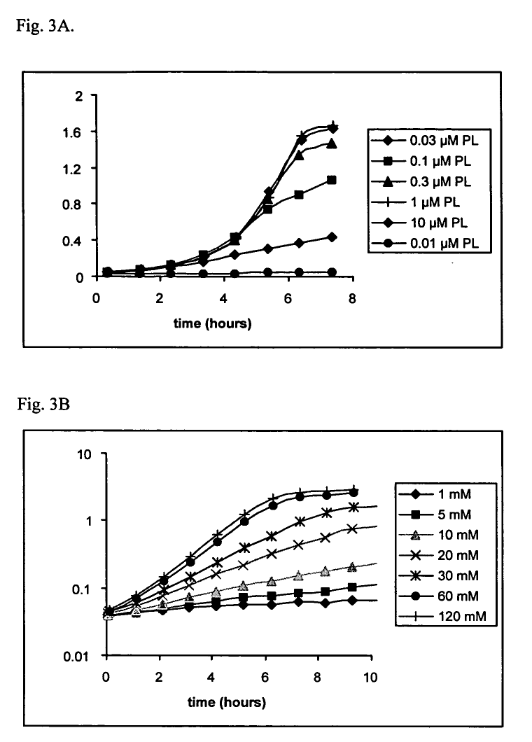

Strain BB2253 (having the yaaD mutation) demonstrated a phenotype of inability to grow in minimal media, or in minimal medium enriched with amino acids. However, growth was fully restored by the addition of 1 μM pyridoxal (PL; FIG. 3A). The discovery herein of a strict requirement for PL in these mutant cells shows that only a single pathway of PLP biosynthesis exists in B. subtilis cells.

Growth of strain BB2253 was only partially restored by 200 μM pyridoxine (PN), indicating that B. subtilis cells fail to utilize exogenous PN for PLP biosynthesis or use PN only weakly. Poor ability to utilize PN has been observed for uncharacterized B. subtilis mutants that appeared to require B₆ vitamers for growth [Pflug, 1978]. B. subtilis cells apparently lack the pdxH-like gene and the corresponding pyridoxine 5′-phosphate oxidase activity [Pflug, 1983] required for conversion of pyridoxine to PLP. Pyridoxamine was several-fold less effective with the mutant here than pyridoxal (probably due to the specificity of pyridoxal kinase required for phosphorylation of all simple B₆ vitamers or to the specificity of the corresponding transport system). Poor growth of this strain was observed in the presence of PLP. Significant growth defects of strain BB2253, relieved by addition of pyridoxal, were also observed in most rich media, including LB-broth, brain heart infusion medium or DS medium, indicating that these media lack a sufficient amount of B₆ vitamers to support full growth of the null yaaD mutant.

Strain BB2256 (yaaE) demonstrated a phenotype in minimal medium of conditional PL auxotrophy, depending on ammonium availability. Virtually no growth was observed in a medium having glucose-glutamate, or in glucose-1 mM ammonium media. Further, growth of this strain at a wild-type rate was observed in medium supplemented with ammonium salt at a concentration of 60-120 mM (FIG. 3). Good growth was observed in glucose-30 mM ammonium medium, although at a rate slower than that of cells grown with 60-120 mM ammonium salts. Without being limited by any particular mechanism, these growth characteristics of the yaaE mutant show that YaaE protein encodes a glutaminase subunit of a glutamine amidotransferase, a synthase subunit of which uses ammonium as an alternative substrate. Finally, while partial growth defects were observed in rich media, the growth rate of strain BB2256 under any of these conditions was restored to that of the wild type cell in the presence of PL.

It is herein established that previously uncharacterized genes yaaD and yaaE are involved in B₆ synthesis, and hence these genes are here renamed pdxS and pdxT, respectively. A strain (BB2254) carrying a deletion of parts of both pdxS and pdxT genes was constructed, and was found to have the same phenotype as a single pdxS null mutant. No revertants or pseudorevertants (suppressed by second site mutations) of such mutants have been observed in spite of a search of a large number of cells for reversion to prototrophy. Because of the finding that each of the pdxS and pdxST deletions cause a requirement for B₆ vitamers, it is here shown that there is only one functional pathway of de novo PLP synthesis in B. subtilis, and most likely in other bacteria possessing the pdxST-like genes. The existence of a PdxS/PdxT-dependent pathway in PLP synthesis has not previously been shown for any bacterium.

These data show that the products of the pdxS and pdxT genes are enzymatic members of the pathway of de novo PLP synthesis in B. subtilis. In contrast in E. coli, reactions that are capable of bypassing several normally functioning steps of the de novo PLP pathway have been reported [Lam, 1990, Man, 1996]. Unlike in E. coli, inability herein to isolate phenotypic revertants (i.e., cells carrying second site suppressing mutations) of the null pdxS mutant in B. subtilis indicates that at least this step is an essential and singular part of the PLP synthesis pathway.

A Bacillus circulans pdxT mutant gene was described however, no function of the gene was ever determined. A partial growth defect dependent on ammonium availability was also reported for the mutant [Tamegai, 2002]. The growth defect was not remediated by addition of pyridoxine.

No effect of isoleucine addition (500 μg/ml), with or without pyridoxine, was here observed on growth of the B. subtilis pdxS and pdxT mutants described herein. Therefore these mutants are different from B. subtilis apparent B₆-dependent mutants [Pflug, 1978].

Insertion mutations in what is here shown to be pdxS and pdxT genes were previously isolated as part of a large project for systematic inactivation of B. subtilis genes (http://bacillus.genome.adjp), however these mutations were not further characterized. No function of these genes has been suggested. The mutants in that study grow in rich medium, though at a reduced rate (http://bacillus.genome.adjp), giving no indication of the function of those genes.

### Example 2

### Construction of a Conditional pdxs Mutant

A pdxS gene lacking its own promoter was synthesized by PCR containing the using SMY chromosomal DNA as template and pdxS-specific primers, oBB138 (5′-ATAACGGATCCTTGATTAGGGGGACC; SEQ ID NO: 9) and oBB139 (5′-TCAGCCGCGGCTCCTATGTTCTTACC; SEQ ID NO: 10), containing the BamHI and SacII sites (in bold), to obtain a 0.92-kb fragment. The fragment was cloned in pAX01 [Härt1, 2001], a vector containing a tightly-repressed xylose-inducible Pxyl promoter and conferring erythromycin resistance, to create plasmid pBB1249.

Strain BB2281 carrying the Pxyl-pdxS construct integrated at the lacA locus was isolated by transforming strain BB1263 (lacA::spc) with pBB1249, selecting for resistance to erythromycin, and screening for loss of spectinomycin-resistance. Selection for this combination of markers yields cells having a phenotype that is a result of a double crossover, homologous recombination event to yield a pdxS null mutant containing the Pxyl-pdxS construct (strain BB2282). Cells of this strain were found to require xylose for growth in minimal medium as an inducer of the inserted construct. Further, these cells were capable of growth in the absence of xylose in B₆-containing medium, due to activity of a salvage pathway.

These data show that strains have been constructed to have conditional expression of a pdx gene, for example, apdx gene that is inducible by xylose, and that this expression is useful to overcome growth defects of mutant strains described herein during culture, for example, of strains carrying mutations such aspdxS, pdxZ, or even of double mutants such aspdxSpdxZ.

### Example 3

### Purification of PdxS and PdxT

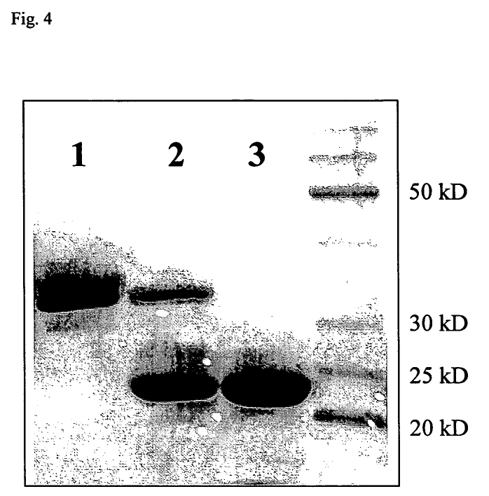

A His₆-tagged B. subtilis PdxS protein, encoded as described herein, was overproduced in E. coli cells, and was purified to virtual homogeneity using Ni²⁺-affinity chromatography (FIG. 4, lane 1). The predicted molecular mass of the unmodified protein is 31.5 kD. A His₆-tagged version of B. subtilis PdxT (of predicted molecular mass for the unmodified protein 21.3 kD) was purified herein in a similar manner (FIG. 4, lane 3). The modified proteins were expressed in B. subtilis cells, and were found to be active and to complement null mutations in cells carrying the corresponding chromosomal mutated alleles as shown herein.

### Example 4

### PdxS and PdxT Interact Physically

E. coli strains overproducing each of the wild-type version of the B. subtilis PdxS protein and the His₆-tagged version of PdxT were constructed, and were used for purification of the PdxT-His₆ protein by Ni²⁺-affinity chromatography.

Wild-type PdxS, which per se has no affinity for a Ni²⁺-affinity column, was found to copurify with PdxT-His₆, though not in an equimolar stoichiometric amount. These data indicate that the two B. subtilis proteins form a complex in vivo which does not completely dissociate even under the high salt conditions (0.5 M NaCl, 0.4 M imidazole) used for purification (FIG. 4, lane 2).

### Example 5

### PdxS and PdxT form a Protein Complex Having Glutaminase Activity

The organization and sequence of the pdxT gene indicates that it is unlikely to encode a glutamine amidotransferase. It was therefore envisioned that pdxT is most probably a gene for a glutaminase subunit of a heteromultimeric glutamine amidotransferase.

Glutamine amidotransferases are enzymes that utilize the amide nitrogen of glutamine and incorporate it into diverse compounds. All glutamine amidotransferases are composed of either two different subunits, or of two functional domains having each of a glutaminase and a synthase activity, respectively. Glutaminase subunits/domains are responsible for hydrolysis of glutamine to glutamate and ammonia. The ammonia is channeled to the corresponding synthase subunits/domain and is incorporated into a compound as is determined by the specificity of the particular glutamine amidotransferase [Zalkin, 1998].

The genetic location ofpdxS adjacent to pdxT makes pdxS a candidate for a gene encoding the synthase subunit of a glutamine amidotransferase of a novel of an as yet unknown specificity. The phenotypes of null pdxS and pdxT mutants observed herein (strict requirement or conditional requirement for B₆ vitamers, respectively) support this interpretation. Most glutamine amidotransferases utilize ammonium (though with low efficiency) instead of glutamine. The glutaminase subunit is completely dispensable for ammonium utilization (the real substrate of glutamine amidotransferases is ammonia which is in equilibrium with ammonium in aqueous solution). Inactivation of glutaminase subunits of several other glutamine amidotransferases leads to conditional (ammonium-dependent) phenotypes of corresponding mutants [Zalkin, 1993]. Finally, the interaction demonstrated herein between PdxS and Pdx t is consistent with the hypothesis that the two proteins are subunits of a single enzymatic entity. The reaction catalyzed by the PdxST complex is not yet known. In the absence of such knowledge, a known partial activity of glutamine amidotransferases, the glutaminase reaction which frequently occurs in the absence of other substrates [Zalkin, 1993] was tested.

Glutaminase activity was here detected in a reaction coupled to reduction of 3-acetylpyridine analog of NAD (shifts the unfavorable equilibrium of the coupled reaction) and conversion of glutamate to 2-ketoglutarate catalyzed by bovine glutamate dehydrogenase. Purified PdxT per se had very little glutaminase activity if glutamine was provided as the only substrate (FIG. 5); PdxS per se had no glutaminase activity. When PdxS was present together with PdxT (in the same ratio as in FIG. 4, lane 2), much higher glutaminase activity was observed (FIG. 5). Even higher activity was obtained by increasing the ratio of PdxS/PdxT complex. This result strongly confirms the present model that PdxS and PdxT are subunits of the same enzyme. Further, the growth phenotype of the pdxT mutant indicates the ammonium-dependent activity of PdxS in the absence of its cognate glutaminase subunit.

In vivo in an infected subject, a concentration of ammonium ions is insufficient to support bacterial growth in the absence of a functional PdxT. Therefore, growth defects that are conferred by mutation on pdxS and pdxT mutants are manifested in infected organisms. Hence these strains are suitably attenuated for use in a vaccine.

### Example 6

### Operon Structure and Regulation of Expression of pdxST Genes

ApdxS-lacZ transcriptional fusion (plasmid pBB1259; FIG. 2) was constructed as described in Materials and Methods herein, and was integrated at the ectopic amyE locus of the B. subtilis chromosome (strain BB2279).

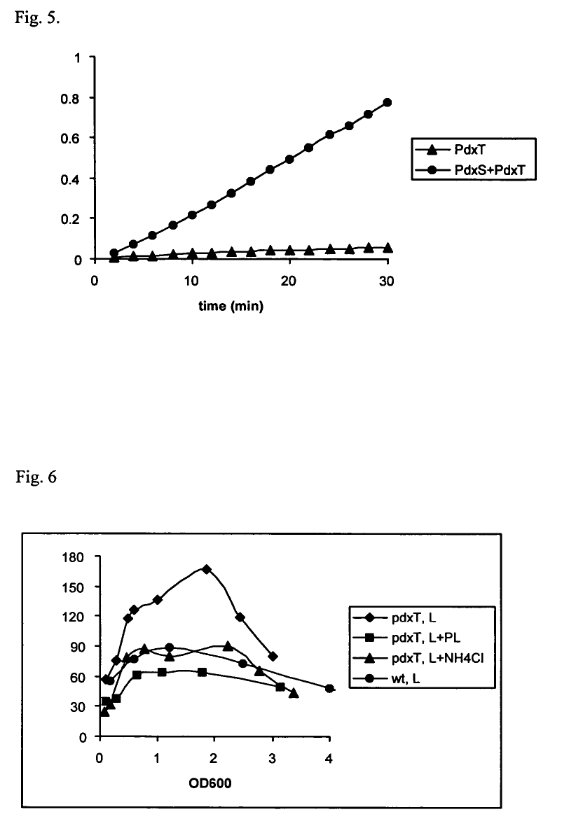

The pdxS-lacZ fusion construct was found to be expressed at the level of 100-200 Miller units of β-galactosidase activity, in cells grown in TSS-glucose medium with ammonium or with glutamate as nitrogen source (Table 5), and also during late exponential growth in DS nutrient broth medium. No effect of addition of excess pyridoxal (up to 1 mM) was observed on expression or on growth rate. Further, a moderate activation of expression (about 3-4-fold) was observed as a result of B₆ limitation, for example, by growth of apdxT mutant in LB broth. This effect was not observed or was much less pronounced for the pdxT mutant using LB broth supplemented with excess PL or NH₄Cl (FIG. 6), or in a wild type strain. Further, a 2-3-fold decrease in β-galactosidase activity was observed inpdxT mutant cells grown in TSS-glucose mediume with limiting concentrations of ammonium salts, and this decrease in pdxS-lacZ expression was prevented by the presence of PL (Table 5).

A pdxST-lacZ transcriptional fusion (plasmid pBB1254; FIG. 2) was constructed, and was shown to be expressed at the ectopic amyE locus in strain BB2277 at a level of 29 Miller units of P-galactosidase specific activity in TSS-glucose-ammonium medium. Deleting a sequence upstream of the BseRI site from this fusion, which comprises the pdxS promoter (plasmid pBB1262) reduced expression of P-galactosidase in the corresponding strain BB2285 about 150-fold (to 0.2 Miller units). Thus, expression of the pdxT gene depends on function of the pdxS promoter, indicating that pdxS and pdxT form an operon. Further, the pdxST-lacZ fusion construct contains a full length pdxS gene, and was found to fully complement the pdxS null mutation, indicating that the latter is recessive.

Each of the pdxS-lacZ and pdxST-lacZ transcriptional fusions were also integrated separately at the pdxST locus of the B. subtilis chromosome. Such an integration event allows the promoterless lacZ reporter gene to be expressed not only from the pdxS promoter but also due to possible read-through from the upstream dacA promoter. The corresponding strains containing these constructs, BB2280 and BB2278, were found to have β-galactosidase activities about 2-fold higher than those of strains described above which contain the same fusions at the ectopic locus, indicating that the dacA is not a predominant promoter for pdxST expression. A pdxS-lacZ translational fusion was constructed (plasmid pBB1263; FIG. 2) and integrated at the pdxS locus. Expression of this fusion in BB2294 cells was similar to the expression pattern described above for the transciptional fusion, a finding that confirms that contribution of the readthrough from the dacA promoter to pdxST expression is not significant.

Expression of the B. subtilis pdxST operon characterized herein does not correlate with B₆ availability. Expression does not respond to PL excess in the medium, and is surprisingly either elevated or reduced, respectively, under conditions in which B₆ limits growth of cells in rich or minimal media, respectively. Growth phase-dependent regulation of the pdxST operon was observed in DS nutrient broth medium. No carbon or nitrogen catabolite regulation and no autoregulation was detected. In an earlier report, expression of PdxS (then called YaaD) was induced by superoxide ions [Antelmann, 1997], a class of agents which is known to activate the σ^{B}-regulon, although no dependence ofpdxS expression on σ^{B}-RNA polymerase was detected [Price, 2001].

PdxS-like proteins are involved in fungal resistance to singlet oxygen species [Ehrenshaft, 1998; Padilla, 1998; Osmani, 1999] and to superoxide [Rodriguez-navarro, 2002] and in general stress response in plants [Sivasubramaniam, 1995]. Pyridoxine and its derivatives were identified as potent quenchers of singlet oxygen in vitro [Ehrenshaft, 1998; Bilski, 2000]. Additionally, pyridoxamine was shown to be a scavenger of reactive carbonyl intermediates of carbohydrate and lipid degradation [Voziyan, 2002] and to prevent superoxide radical formation [Jain, 2001].

Without being limited by any particular theory or molecular mechanism, while the precise mechanism of pdxST regulation is unknown, the examples herein indicate that it may be similar to B₆-independent modes of regulation of pdx genes in E. coli [Man, 1997; Pease, 2002] and in yeast [Padilla, 1998; Rodriguez-Navarro, 2002]. Some predicted genes of the de novo pathway of PLP synthesis in bacteria appear to be regulated by dedicated PLP-containing transcriptional regulators of the MocR/GabR family [Belitsky, 2002] encoded by adjacent divergent genes (Table 2).

### Example 7

### Analysis of Pathways of De Novo PLP Biosynthesis in Bacteria with Sequenced Genomes

Published genome sequence data were searched by using the NCBI Blast server at http://www.ncbi.nlm.nih.gov/BLAST. Preliminary sequence data were searched at http://www.ncbi.nlm.nih.gov/cgi-bin/Entrez/genom_table_cgi, http://tigrblast.tifr.org/ufing/, http://www.sanger.ac.uk/DataSearch/, and http://artedi.ebc.uu.se/Projects/Francisella/blast/, http://hgsc.bcm.tmc.edu/microbial/microbialblast.cgi?organism=Mhaemolytica, and http://genome3.cpmc.columbia.edu/˜legion/int_blast.html. The Blast program [Altschul, 1997] was used for genomic searches.

Table 2 shows that no single bacterial genome has genes encoding all of the four proteins, PdxA, PdxJ (used here as representative of the E. coli-like pathway), PdxS (YaaD), and PdxT (YaaE). Some bacteria with the PdxST pathway have a PdxA homolog; it is likely that these orphan PdxA-like proteins, as well as additional PdxA paralogs in some gram-negative bacteria (e.g., Agrobacterium tumefaciens), have a dehydrogenase activity that is unrelated to PLP synthesis. Most Gram-negative bacteria are here shown to have the PdxA/PdxJ pathway; Gram-positive bacteria have either the PdxST pathway or none. Some lactic acid bacteria and clostridia are known to be B₆ auxotrophs [Snell, 1989; Mulligan, 1977; Boyd, 1948] consistent with the absence of genes of de novo PLP synthesis in their genomes. ThepdrS andpdxT-like genes are linked in all bacterial genomes other than Mycobacterium leprae.

No gene similar to dxs (FIG. 1) was found in searches of the complete genomes of Staphylococcus aureus or Streptococcus pneumoniae, both of which from the analysis in Table 2 are likely to utilize the PdxST pathway. Therefore, 1-deoxy-D-xylulose-5-phosphate, the product of the Dxs-catalyzed reaction, is an unlikely intermediate in the PdxST-dependent pathway of PLP synthesis.

PdxS may have a phosphate-binding site [Galperin, 1997] implicating a phosphorylated compound as a substrate; another possible role for this site is binding of FMN (the IPR003009 motif at www.ebi.ac.uk/interpro). In the analysis herein, it was observed that many enzymes containing an IPR003009 motif do not bind FMN, rather these enzymes have pent(ul)ose 5-phosphate derivatives as their substrates. Moreover, FMN itself contains a ribityl-5-phosphate group. Therefore, it is likely that the IPR003009 motif in proteins is asociated with binding of a five-carbon unit phosphorylated at the position 5. Thus a substrate of PdxST, in addition to glutamine, may be a phosphorylated five-carbon carbohydrate derivative.

As shown previously [Pflug, 1978] and in this work, B. subtilis cells can utilize exogenous simple B₆ vitamers to satisfy their requirement for PLP. The fact of relative preferential utilization of PL compared to poorly used PN indicates that B. subtilis cells lack a pdxH-like gene and encoded pyridoxine 5′-phosphate oxidase activity [Pflug, 1983] which is required for conversion of pyridoxine to PLP. Lack of a pdxH gene in the bacterial genomes observed herein, including essentially all Gram positive bacteria (Table 2) indicates that these strains would be unable to oxidize pyridoxine to pyridoxal, and therefore unable to use pyridoxine. For example, several lactic acid bacteria lack pdxH and the genes for the de novo PLP synthesis as shown by their genome analysis(Table 2), and these strains use pyridoxal preferentially [Snell, 1989]. A list of strains having PdxS-like genes as determined by genomic searching is shown in Table 3.

A correlation between a presence of a de novo PLP synthesis pathway and a presence of at least one PdxH or PdxK/PdxY homolog was sought (Table 2) but was not observed. No genes similar to pdxK or pdxY were found in genomes of most Gram-positive bacteria or in many Gram-negative bacteria for which genomic data is available. Lack ofpdxK-like genes among these genomes indicates that the primary structure of B₆ kinases in these strains differs from the amino acid sequence of E. coli PdxK.

### Example 8

### Identification of an Essential Gene of the Salvage Pathway of PLP Synthesis in B. subtilis

A novel type of pyridoxal kinase that is required for the salvage pathway of PLP synthesis was identified as described herein. Designated herein as PdxZ (previously misidentified as ThiD, and also annotated YwdB), this enzyme belongs to the phosphomethylpyrimidine kinase family of proteins (IPR004399, COG0351), and is different from members of the pyridoxal kinase family (IPR004625, COG2240) to which mammalian and E. coli enzymes belong.

E. coli PdxK pyridoxal kinase phosphorylates both B₆ vitamers and an intermediate of thiamine synthesis [Mizote, 1989; Reddick, 1998; Mizote, 1999]. The latter can also be phosphorylated by the product of the E. coli thiD gene, an essential enzyme of thiamine synthesis with activities of 4-amino-5-hydroxymethyl-2-methylpyrimidine and 4-amino-5-hydroxymethyl-2-methylpyrimidine phosphate kinases [Begley, 1999]. It seemed possible that in some bacterial strains a ThiD-like protein may have a pyridoxal kinase activity. There are two thiD-like genes in the B. subtilis genome [Kunst, 1997]. One of these is identified as ywdB, presently misannotated as thiD, and is in fact a gene of unknown function. The other has been identified as yjbV, and as it is located among genes of thiamine biosynthesis may be the true thiD ortholog [Begley, 1999]. It is shown herein that ywdB (identified herein as pdxZ) is involved in B₆ salvage.

A pdxZ null mutant of wild-type B. subtilis strain SMY was constructed by replacing a part of the pdxZ gene cloned on a plasmid with an antibiotic resistance cassette (spc gene conferring spectinomycin resistance), and replacing the chromosomal allele of the pdxZ gene with the mutant plasmid copy due to a double-crossover homologous recombination event after transformation, to yield strain BB2251. Cells of strain BB2251 (pdxZ::spc) had wild-type growth characteristics.

Double pdxS pdxZ and pdxT pdxZ null mutants, defective both in de novo and salvage pathways of PLP synthesis, were obtained by introducing the pdxZ mutation into the chromosome of the pdxS or pdxT mutant cells by transformation. The double mutant pdxS pdxZ (strain BB2267) was found to be non-viable when plated or streaked on DS nutrient broth medium or glucose-ammonium minimal salts TSS medium supplemented with 1 μM pyridoxal or 200 μM pyridoxine, due to inability of isolated cells to utilize exogenous PLP precursors even at concentrations sufficient to support growth of cells carrying either a pdxS or pdxT mutation.

Growth and viability of cells of this double mutant were found herein to be obtained by supplementation of growth medium with a very high concentration (1 mM) of pyridoxal, i.e., with a non-physiological concentration which exceeds by 1000-fold the concentration of pyridoxal required for full growth of the pdxS mutant. Similar results were obtained by addition of 1 mM of PLP. Viability of cells of strain BB2295 carrying both pdxS and pdxZ mutations was also obtained in the absence of exogenous pyridoxal in the presence of xylose, by conditionally restoring the de novo pathway of PLP synthesis with a xylose-inducible form of the pdxS gene at an ectopic location, i.e., located elsewhere on the chromosome. These and similarly constructed conditional lethal strains can be used for vaccine development, to overcome possible lethality to the bacteria during culture of cells carrying double pdxS and pdxZ or double pdxT and pdxZ mutations.

A similar result was obtained with a strain having double pdxT pdxZ null mutations: this strain had a conditional lethal phenotype, and cells were found to be viable in medium having high ammonium concentrations but did not grow even in the presence of physiological concentrations of PLP precursors in media containing a low concentration of ammonium (e.g., DS nutrient broth medium or glucose-glutamate minimal salts TSS medium).

Examples herein show that ywdB (also previously misidentified as thiD, and designated herein pdxZ) encodes a pyridoxal kinase required for the salvage pathway of PLP synthesis in B. subtilis. While PdxZ is similar to ThiD-like proteins, PdxK is only distantly related to ThiD proteins; as these proteins belong to two different member families of the same PfkB superfamily of kinases [Wu, 1991]. A Blast search comparison [Altschul, 1997] indicates that B. subtilis PdxZ and E. coli PdxK are less than 25% identical, having a score of 59.3 bits and an expect value of 3xe⁻⁰⁹. Further, E. coli PdxY and mammalian pyridoxal kinases are even less similar to B. subtilis PdxZ (score=45.8 bits and an expect value of 7xe⁻⁰⁴). Thus, the structure of PdxZ is sufficiently different from the structure of mammalian pyridoxal kinase to provide herein that B. subtilis PdxZ serves as a promising drug target for identification of novel antibacterial agents.

A list of bacterial strains in which the present genomic searches have identified apdxZ-like gene in the absence of any pdxK-like gene is shown in Table 4. Use of PdxZ as a target, for identification of an antibacterial agent capable of inhibiting PdxZ, may be useful to obtain novel antibacterial antibiotic drugs useful for treating pathogenic Gram positive and negative bacteria in animal subjects including humans, and also antibacterial agents for crop protection from bacterial plant pathogens such as Raistonia solanacearum and Xylella fastidiosa. Further use of an inhibitor of PdxZ may be as an antibacterial agent for insecticidal application, for example, against a bacterial symbiont Wigglesworthia brevipalpis found in tsetse fly, and essential for insect growth.

In some pathogenic bacteria such as Enterococcus faecalis or Enterococcus faecium, the PdxZ-dependent salvage pathway is the only pathway of PLP biosynthesis. Inhibition of this pathway by anti-PdxZ compounds is expected to inhibit growth of such wild type bacteria under all conditions of growth. These strains may be used as test strains for identification of potential antibacterial agents that target the PdxZ protein.

### Example 9

### Analysis of Pathways of Salvage PLP Biosynthesis in Bacteria with Sequenced Genomes

Published genome sequence data were searched for identification of genes encoding enzymes of PLP salvage pathways, by using the NCBI Blast server at http://www.ncbi.nlm.nih.gov/BLAST. Preliminary sequence data were searched at http://www.ncbi.nlm.nih.gov/cgi-bin/Entrez/genom_table_cgi, http://tigrblast.tifr.org/ufing/, http://www.sanger.ac.uk/DataSearch/, and http://artedi.ebc.uu.se/Proiects/Francisella/blast/, http://hgsc.bcm.tmc.edu/microbial/microbialblast.cgi?organism=Mhaemolylica and http://genome3.cpmc.columbia.edu/·legion/int_blast.html. The Blast program [Altschul, 1997] was used for genomic searches.

Results of these searches (Table 2) show that no genes similar to E. coli genes paxK or pdxY, encoding pyridoxal kinases, which are essential enzymes of the PLP salvage pathway (FIG. 1), were found in genomes of most Gram-positive bacteria or in many Gram-negative bacteria for which genomic data is available. Lack of pdxK-like genes among these genomes indicates that the primary structure of B₆ kinases in these strains differs from the amino acid sequence of E. coli PdxK. A novel pyridoxal kinase, B. subtilis PdxZ, was identified in examples herein. A list of bacterial strains in which the present genomic searches have identified a pdxZ-like gene and no pdxK-like gene is shown in Tables 2 and 4. These searches also show that some bacteria have neither a PdxK/PdxY-type pyridoxal kinase, nor a PdxZ-type pyridoxal kinase. Further, some of the searched bacteria, such as Clostridium tetani and Borrelia burgdorferi, contain still another type of pyridoxal kinase (marked as PdxK# in Table 2), which occupies an intermediate position between mammalian and E. coli-like pyridoxal kinases on one hand, and B. subtilis PdxZ-like pyridoxal kinase on the other.

While PdxK# proteins belong to the same PfkB superfamily of kinases [Wu, 1991] as PdxK/Y, PdxZ, and mammalian pyridoxal kinases, PdxK# proteins are only distantly related to other pyridoxal kinases. A Blast search comparison [Altschul, 1997] indicates that C. tetani PdxK# and E. coli PdxK are less than 24% identical, having a score of 53.5 bits and an expect value of 4xe⁻⁰⁶. Further, E. coli PdxY, B. subtilis PdxZ and mammalian pyridoxal kinases are even less similar to C. tetani PdxK# (identity to human pyridoxal kinase is less than 23% with a score=46.6 bits and an expect value of 5xe⁻⁰⁴). Thus, the structure of a PdxK#-like protein is sufficiently different from the structure of a mammalian pyridoxal kinase to use the PdxK#-like protein as a drug target for identification of novel antibacterial agents.

A list of bacterial strains in which the present genomic searches have identified a pdxK#-like gene and no pdxK/pdxY-like or pdxZ-like genes is included in Table 4. Methods of screens herein that use a PdxK# protein as a target may yield broad spectrum antibacterial antibiotic drugs, i.e., drugs that can be useed to treat infections by pathogenic bacteria that are Gram positive or Gram negative. In some species of pathogenic bacteria, such as Clostridium botulinum, C. perfringens, C. difficile, C. tetani, Streptococcus pyogenes, and S. mutans, the PdxK#-dependent salvage pathway is the only pathway of PLP biosynthesis. Inhibition of this pathway in such bacterial species by an anti-PdxK# compound is expected to inhibit such bacteria under any condition of growth.

### Example 10

### Isolation and Overexpression of Geobacillus stearothermophilus Pdxs and PdxT

A sequence of a chromosomal fragment containing the pdxS and pdxT genes of G. stearothermophilus (previously known as Bacillus stearothermophilus) strain 10 was obtained through the Bacillus stearothermophilus Genome Sequencing Project at www.genome.ou.edu/bstearo_blast.html. A 0.95-kb DNA fragment coding for a modified form of PdxS containing a His₆-tag at its C-terminus was synthesized by PCR, using chromosomal DNA of G. stearothermophilus as a template, and oligonucleotides oBB126 (5′-CTTTTGCATGCCTCGCAAAGC; SEQ ID NO: 11), and oBB127 (5′-TTATTAAGCTTAGTGGTGGTGGTGGTGGTGCCAGCCGCGTTCTTGCATC; SEQ ID NO: 12), as 5′- and 3′-primers, respectively (SphI and HindIII sites are shown in bold). The PCR fragment was digested with SphI and HindIII, and was cloned into expression vector pBAD18 containing an inducible E. coli ara promoter [Guzman, 1995] previously digested with SphI and HindIII, to create pBB1186.

Similarly, a plasmid pBB1235 overexpressing a PdxT-His₆ protein was constructed by cloning a 0.65-kb KpnI-XbaI pdxT-containing fragment in the pBAD18 vector [Guzman, 1995]. The pdxT fragment was synthesized by PCR using chromosomal DNA of G. stearothermophilus as a template, and oligonucleotides oBB142 (5′-aaaGGGGTACCGGATGCAAGAACGC; SEQ ID NO: 13), and oBB143 (5′-ATCACTCTAGATTAATGGTGATGGTGATGGTGCTTGAGGCTTGACGCC; SEQ ID NO: 14), as 5′- and 3′-primers, respectively (the KpnI and XbaI sites are in bold).

The G. stearothermophilus PdxS and PdxT proteins were purified as described herein for corresponding B. subtilis proteins. It was found that the G. stearothermophilus PdxST complex has glutaminase activity, assayed as described above for B. subtilis enzyme. Thus, G. stearothermophilus possesses enzymes for the PdxST pathway of de novo PLP synthesis.

### Example 11

### Construction of a pdxST Null Mutant of Listeria monocytogenes Strain EGDe

A fragment containing an insertion of the 1.1-kb spc cassette replacing most (1 kb) of the pdxS (Lmo2101) and pdxT (Lmo2102) genes was generated by an overlapping PCR approach as follows. A 0.9-kb PCR fragment containing the 3-prime end ofpdxS and the upstream region was synthesized using chromosomal DNA of L. monocytogenes as a template, and oligonucleotides oBB130 (5′-CATCACTAGTCCAGTTGGATTTTG SEQ ID NO: 15), and oBB131 (5′-CGCCAGGGCTGCAGGAATTCTCCATATTAATAACCTCCATC SEQ ID NO: 16), as 5′-oBB131 and 3′-primers, respectively (SpeI site is shown in bold, and an overlap with the 5′-end of the spc cassette is underlined).

Further, a 0.9-kb PCR fragment containing the 5-prime end ofpdxT and the downstream region was synthesized using chromosomal DNA of L. monocytogenes as a template, and oligonucleotides oBB132 (5′-GGGAAATATTCATTCTAATTGGATGAATTACTTCCTCG SEQ ID NO: 17), and oBB133 (5′-AAGAAGAGCTCGTAAAACCAATCTTACTTGG SEQ ID NO: 18), as 5′- and 3′-primers, respectively (a SacI site is in bold, and the overlap with the 3′-end of the spc cassette is underlined). The primers oBB131 and oBB132 contained sequences complementary to 5′- and 3′-ends of the spectinomycin-resistance (spc) cassette of pJL74 [LeDeaux, 1995], respectively.

The 0.9-kb products of the first two PCR reactions, containing 20-22 nucleotide overhangs complementary to the ends of the spc cassette originating from the primers oBB131 and oBB132, were used as 5′- and 3′-primers, respectively, in the third PCR reaction with pJL74 as a template. The final 3-kb PCR product contained the spc cassette flanked by the 3′-end of the pdxS gene (and upstream sequences) and the 5′-end of the pdxT gene (and downstream sequences). A SpeI-SacI fragment of this PCR product was cloned between XbaI and SacI sites of vector pCon-1 containing a temperature sensitive replicon of pE194 [Smith, 1992] and pRK2 transfer origin locus.

The resulting plasmid, pBB1212, was introduced into E. coli strain DW1030 containing conjugative plasmid pRK231.7, a broad host range plasmid of the IncP incompatibility group. pBB1212 was transferred from strain DW1030 (pBB1212) into L. monocytogenes cells by conjugation [Trieu-Cuot, 1987] at the temperature (30°) permissive for replication of pBB1212. The ΔpdxST::spc mutants, formed due to allelic exchange resulting from pBB1212 plasmid integration into the chromosome at the non-permissive temperature (41°) and excision at the permissive temperature, were obtained by screening colonies that lost the plasmid marker (chloramphenicol-resistance) and had retained resistance to spectinomycin. Replacement of the chromosomal pdxST locus by the ΔpdxST::spc allele in strain BL1 was confirmed by comparing the sizes of PCR fragments obtained by use of the wild-type and mutant pdxST chromosomal loci as templates.

Strain BL1 (ΔpdrST::spc) was observed to have a growth defect phenotype, comprising poor growth in the brain heart infusion medium and essential inability to grow on LB-plates. Growth of the mutant was fully restored by the presence 1 μM pyridoxal, but not at all by 100 μM pyridoxine. These traits of the L. monocytogenes mutant are similar to those observed herein with the corresponding B. subtilis mutant.

The results show that pathogenic bacterium L. monocytogenes has acquired the PdxST-dependent pathway of de novo PLP synthesis. These data show that an antibiotic capable of targeting PdxST would inhibit growth of cells of species of Bacillus, Geobacillus, and Listeria, and of many other Gram positive species.

### Example 11

### Bacteria Having PdxS, Lacking PdxT, and Glutaminase Activity

Genomes of several bacterial species including Fusobacterium nucleatum, Corynebacterium efficiens, and probably C. botulinum (having an as yet incompletely sequenced genome), have an orphan PdxS-like gene and no PdxT-like gene (Table 2). PdxS proteins of these bacteria may operate as ammonium-dependent synthases of the PLP pathway; other possibilities include dependence on PdxT-unrelated glutaminase subunits, or inactivity of these pdxS-like genes or their products (cryptic genes).

The glutaminase reaction of the PdxST complex described herein can be used as a preliminary test system in a search for potential substrates of the PdxST complex, other than glutamine. It is known that glutaminase activity of several glutamine amidotransferases is stimulated by the corresponding substrates or their close analogs [Zalkin, 1993; Li, 1971]. Further, the glutaminase reaction of the PdxST complex described herein can be used as an in vitro system for identification of potential inhibitors of these enzymes. A thus-obtained potential inhibitor is a potential antibacterial agent, for example, can be orally administered if it is orally absorbed and non-toxic, or can be topically administered. A potential inhibitor can be an antibacterial pesticide, for example, as an antibacterial agent to protect crop vegetables and fruits.

The glutaminase reaction of B. subtilis PdxST complex provides an easy assay to determine the stoichiometry of PdxS and PdxT interaction required for maximal enzymatic activity of the complex. It can further serve as a target for identification of inhibitors that are potential antibacterial therapeutic agents, for a variety of bacterial pathogens as shown in Tables 2 and 3 including Gram positive pathogenic species such as B. anthracis, M tuberculosis, and other Gram positive pathogens, and Gram negative species such as Francisella tularensis, Haemophilus influenzae, and Pasteurella multocida.

### REFERENCES

Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Res. 25:3389-402.
Antelmann, H., J. Bernhardt, R. Schmid, H. Mach, U. Volker, and M. Hecker. 1997. First steps from a two-dimensional protein index towards a response-regulation map for Bacillus subtilis. Electrophoresis 18:1451-63.
Bean, L. E., W. H. Dvorachek, Jr., E. L. Braun, A. Errett, G. S. Saenz, M. D. Giles, M. Werner-Washburne, M. A. Nelson, and D. O. Natvig. 2001. Analysis of the pdx-1 (snz-1/sno-1) region of the Neurospora crassa genome: correlation of pyridoxine-requiring phenotypes with mutations in two structural genes. Genetics 157:1067-75.
Begley, T. P., D. M. Downs, S. E. Ealick, F. W. McLafferty, A. P. Van Loon, S. Taylor, N. Campobasso, H. J. Chiu, C. Kinsland, J. J. Reddick, and J. Xi. 1999. Thiamin biosynthesis in prokaryotes. Arch. Microbiol. 171:293-300.
Belitsky, B. R., M. C. Gustafsson, A. L. Sonenshein, and C. Von Wachenfeldt. 1997. An lrp-like gene of Bacillus subtilis involved in branched-chain amino acid transport. J. Bacteriol. 179:5448-57.
Belitsky, B. R., P. J. Janssen, and A. L. Sonenshein. 1995. Sites required for GltC- dependent regulation of Bacillus subtilis glutamate synthase expression. J. Bacteriol. 177:5686-95.
Belitsky, B. R., and A. L. Sonenshein. 2002. GabR, a member of a novel protein family, regulates utilization of g-aminobutyrate in Bacillus subtilis. Mol. Microbiol. 45:569-83.
Belitsky, B. R., and A. L. Sonenshein. 1998. Role and regulation of Bacillus subtilis glutamate dehydrogenase genes. J. Bacteriol. 180:6298-305.
Bilski, P., M. Y. Li, M. Ehrenshaft, M. E. Daub, and C. F. Chignell. 2000. Vitamin B6 (pyridoxine) and its derivatives are efficient singlet oxygen quenchers and potential fungal antioxidants. Photochem. Photobiol. 71:129-34.
Boyd, J. M., M. A. Logan, and A. A. Tytell. 1948. The growth requirements of Clostridium perfringens (welchii) BP6K. J. Biol. Chem. 174:1013-1025.
Buss, K., C. Drewke, S. Lohmann, A. Piwonska, and E. Leistner. 2001. Properties and interaction of heterologously expressed glutamate decarboxylase isoenzymes GAD(65 kDa) and GAD(67 kDa) from human brain with ginkgotoxin and its 5′-phosphate. J. Med. Chem. 44:3166-74.
Drewke, C., and E. Leistner. 2001. Biosynthesis of vitamin B6 and structurally related derivatives. Vitam. Horm. 61:121-55.
Ehrenshaft, M., P. Bilski, M. Y. Li, C. F. Chignell, and M. E. Daub. 1999. A highly conserved sequence is a novel gene involved in de novo vitamin B6 biosynthesis. Proc. Natl. Acad. Sci. USA 96:9374-8.
Ehrenshaft, M., and M. E. Daub. 2001. Isolation of PDX2, a second novel gene in the pyridoxine biosynthesis pathway of eukaryotes, archaebacteria, and a subset of eubacteria. J. Bacteriol. 183:3383-90.
Ehrenshaft, M., A. E. Jenns, K. R. Chung, and M. E. Daub. 1998. SOR1, a gene required for photosensitizer and singlet oxygen resistance in Cercospora fungi, is highly conserved in divergent organisms. Mol. Cell 1:603-9.
Fouet, A., and A. L. Sonenshein. 1990. A target for carbon source-dependent negative regulation of the citB promoter of Bacillus subtilis. J. Bacteriol. 172:835-44.
Galperin, M. Y., and E. V. Koonin. 1997. Sequence analysis of an exceptionally conserved operon suggests enzymes for a new link between histidine and purine biosynthesis. Mol. Microbiol. 24:443-5.
Gupta, R. N., T. Hemscheidt, B. G. Sayer, and I. D. Spenser. 2001. Biosynthesis of vitamin B₆ in yeast: incorporation pattern of glucose. J. Am. Chem. Soc. 123:11353-9.
Guzman, L. M., D. Belin, M. J. Carson, and J. Beckwith. 1995. Tight regulation, modulation, and high-level expression by vectors containing the arabinose PBAD promoter. J. Bacteriol. 177:4121-30.
Härtl, B., W. Wehrl, T. Wiegert, G. Homuth, and W. Schumann. 2001. Development of a new integration site within the Bacillus subtilis chromosome and construction of compatible expression cassettes. J. Bacteriol. 183:2696-9.
Hill, R. E., and I. D. Spenser. 1996. Biosynthesis of vitamin B₆, p. 695-703. In F. C. Neidhardt, R. I. Curtiss, J. L. Ingraham, E. C. C. Lin, K. B. Low, B. Magasanik, W. S. Reznikoff, M. Riley, M. Schaechter, and H. E. Umbarger (ed.), Escherichia coli and Salmonella typhimurium: cellular and molecular biology. American Society for Microbiology, Washington, D.C.
Jain, S. K., and G. Lim. 2001. Pyridoxine and pyridoxamine inhibits superoxide radicals and prevents lipid peroxidation, protein glycosylation, and (Na⁺+K⁺)-ATPase activity reduction in high glucose-treated human erythrocytes. Free Radic. Biol. Med. 30:232-7.
John, R. A. 1995. Pyridoxal phosphate-dependent enzymes. Biochim. Biophys. Acta 1248:81-96.
Kunst, F., N. Ogasawara, I. Moszer, A. M. Albertini, G. Alloni, V. Azevedo, M. G. Bertero, P. Bessieres, A. Bolotin, S. Borchert, R. Borriss, L. Boursier, A. Brans, M. Braun, S. C. Brignell, S. Bron, S. Brouillet, C. V. Bruschi, B. Caldwell, V. Capuano, N. M. Carter, S. K. Choi, J. J. Codani, I. F. Connerton, A. Danchin, and et al. 1997. The complete genome sequence of the gram-positive bacterium Bacillus subtilis. Nature 390:249-56.
Lam, H. M., and M. E. Winkler. 1990. Metabolic relationships between pyridoxine (vitamin B6) and serine biosynthesis in Escherichia coli K-12. J. Bacteriol. 172:6518-28.
LeDeaux, J. R., and A. D. Grossman. 1995. Isolation and characterization of kinC, a gene that encodes a sensor kinase homologous to the sporulation sensor kinases KinA and KinB in Bacillus subtilis. J. Bacteriol. 177:166-75.
Li, H. C., and J. M. Buchanan. 1971. Biosynthesis of the purines. 33. Catalytic properties of the glutamine site of formylglycinamide ribonucleotide amidotransferase from chicken liver. J. Biol. Chem. 246:4713-9.
Mahillon, J., and M. Chandler. 1998. Insertion sequences. Microbiol. Mol. Biol. Rev. 62:725-74.
Man, T. K., A. J. Pease, and M. E. Winkler. 1997. Maximization of transcription of the serC (pdxF)-aroA multifunctional operon by antagonistic effects of the cyclic AMP (cAMP) receptor protein-cAMP complex and Lrp global regulators of Escherichia coli K-12. J. Bacteriol. 179:3458-69.
Man, T. K., G. Zhao, and M. E. Winkler. 1996. Isolation of a pdxJ point mutation that bypasses the requirement for the PdxH oxidase in pyridoxal 5′-phosphate coenzyme biosynthesis in Escherichia coli K-12. J. Bacteriol. 178:2445-9.
Marck, C. 1988. ‘DNA Strider’: a ‘C’ program for the fast analysis of DNA and protein sequences on the Apple Macintosh family of computers. Nucleic Acids Res. 16:1829-36.
Miller, J. H. 1972. Experiments in molecular genetics. Cold Spring Harbor Laboratory, Cold Spring Harbor, N.Y.
Mittenhuber, G. 2001. Phylogenetic analyses and comparative genomics of vitamin B6 (pyridoxine) and pyridoxal phosphate biosynthesis pathways. J. Mol. Microbiol. Biotechnol. 3:1-20.
Mizote, T., and H. Nakayama. 1989. Purification and properties of hydroxymethylpyrimidine kinase from Escherichia coli. Biochim. Biophys. Acta 991:109-13.
Mizote, T., M. Tsuda, D. D. Smith, H. Nakayama, and T. Nakazawa. 1999. Cloning and characterization of the thiD/J gene of Escherichia coli encoding a thiamin-synthesizing bifunctional enzyme, hydroxymethylpyrimidine kinase/phosphomethylpyrimidine kinase. Microbiology 145:495-501.
Mulligan, J. H., and E. E. Snell. 1977. Transport and metabolism of vitamin B6 in lactic acid bacteria. J. Biol. Chem. 252:835-9.
Osmani, A. H., G. S. May, and S. A. Osmani. 1999. The extremely conserved pyroA gene of Aspergillus nidulans is required for pyridoxine synthesis and is required indirectly for resistance to photosensitizers. J. Biol. Chem. 274:23565-9.
Padilla, P. A., E. K. Fuge, M. E. Crawford, A. Errett, and M. Werner-Washburne. 1998. The highly conserved, coregulated SNO and SNZ gene families in Saccharomyces cerevisiae respond to nutrient limitation. J. Bacteriol. 180:5718-26.
Pease, A. J., B. R. Roa, W. Luo, and M. E. Winkler. 2002. Positive growth rate-dependent regulation of the pdxA, ksgA, and pdxB genes of Escherichia coli K-12. J. Bacteriol. 184:1359-69.
Pflug, W., and F. Lingens. 1983. Occurrence of pyridoxamine-phosphate oxidase and pyridoxal kinase in Gram-negative and Gram-positive bacteria. Hoppe Seylers Z. Physiol. Chem. 364:1627-30.
Pflug, W., and F. Lingens. 1978. Vitamin-B6-biosynthese in Bacillus subtilis. Hoppe Seylers Z. Physiol. Chem. 359:559-70.
Price, C. W., P. Fawcett, H. Ceremonie, N. Su, C. K Murphy, and P. Youngman. 2001. Genome-wide analysis of the general stress response in Bacillus subtilis. Mol. Microbiol. 41:757-74.
Reddick, J. J., C. Kinsland, R. Nicewonger, T. Christian, M. E. Diana M. Downs, M. E. Winkler, and T. P. Begley. 1998. Overexpression, purification and characterization of two pyrimidine kinases involved in the biosynthesis of thiamin: 4-amino-5-hydroxymethyl-2-methylpyrimidine kinase and 4-amino-5-hydroxymethyl-2-methylpyrimidine phosphate kinase. Tetrahedron 54:15983-15991.
Rodriguez-Navarro, S., B. Llorente, M. T. Rodriguez-Manzaneque, A. Ramne, G. Uber, D. Marchesan, B. Duj on, E. Herrero, P. Sunnerhagen, and J. E. Perez-Ortin. 2002. Functional analysis of yeast gene families involved in metabolism of vitamins B₁ and B₆. Yeast 19:1261-76.
Sambrook, J., E. F. Fritsch, and T. J. Maniatis. 1989. Molecular cloning: a laboratory manual, 2 ed. Cold Spring Harbor Labroratory, Cold Spring Harbor, N.Y.
Sanger, F., S. Nicklen, and A. R. Coulson. 1977. DNA sequencing with chain-terminating inhibitors. Proc. Natl. Acad. Sci. USA 74:5463-7.
Seack, J., S. Perovic, V. Gamulin, H. C. Schroder, P. Beutelmann, I. M. Muller, and W. E. Muller. 2001. Identification of highly conserved genes: SNZ and SNO in the marine sponge Suberites domuncula: their gene structure and promoter activity in mammalian cells. Biochim. Biophys. Acta 1520:21-34.
Sivasubramaniam, S., V. M. Vanniasingham, C. T. Tan, and N. H. Chua. 1995. Characterisation of HEVER, a novel stress-induced gene from Hevea brasiliensis. Plant Mol. Biol. 29:173-8.
Smith, K., and P. Youngman. 1992. Use of a new integrational vector to investigate compartment-specific expression of the Bacillus subtilis spoIIM gene. Biochimie 74:705-11.
Snell, E. E. 1989. Nutrition research with lactic acid bacteria: a retrospective view. Annu. Rev. Nutr. 9:1-19.
Tamegai, H., E. Nango, A. Koike-Takeshita, F. Kudo, and K. Kakinuma. 2002. Significance of the 20-kDa subunit of heterodimeric 2-deoxy-scyllo-inosose synthase for the biosynthesis of butirosin antibiotics in Bacillus circulans. Biosci. Biotechnol. Biochem. 66:1538-45.
Tanaka, K., K. Tazuya, K. Yamada, and H. Kumaoka. 2000. Biosynthesis of pyridoxine: origin of the nitrogen atom of pyridoxine in microorganisms. J. Nutr. Sci. Vitaminol. 46:55-7.
Trieu-Cuot, P., C. Carlier, P. Martin, and P. Courvalin. 1987. Plasmid transfer by conjugative from Escherichia coli to gram-positive bacteria. FEMS Microbiol. Lett. 48:289-94.
Voziyan, P. A., T. O. Metz, J. W. Baynes, and B. G. Hudson. 2002. A post-Amadori inhibitor pyridoxamine also inhibits chemical modification of proteins by scavenging carbonyl intermediates of carbohydrate and lipid degradation. J. Biol. Chem. 277:3397403.
Wu, L. F., A. Reizer, J. Reizer, B. Cai, J. M. Tomich, and M. H. Saier, Jr. 1991. Nucleotide sequence of the Rhodobacter capsulatus fruK gene, which encodes fructose-1-phosphate kinase: evidence for a kinase superfamily including both phosphofructokinases of Escherichia coli. J. Bacteriol. 173:3117-27.
Yanisch-Perron, C., J. Vieira, and J. Messing. 1985. Improved M13 phage cloning vectors and host strains: nucleotide sequences of the M13mp18 and pUC19 vectors. Gene 33:103-19.
Zalkin, H.1993. The amidotransferases. Adv. Enzymol. Relat. Areas Mol. Biol. 66:203-309.
Zalkin, H., and J. L. Smith. 1998. Enzymes utilizing glutamine as an amide donor. Adv. Enzymol. Relat. Areas Mol. Biol. 72:87-144.

Zeidler, J., N. Ullah, R. N. Gupta, R. M. Pauloski, B. G. Sayer, and I. D. Spenser. 2002. 2′-hydroxypyridoxol, a biosynthetic precursor of vitamins B₆ and B₁ in yeast. J. Am. Chem. Soc. 124:4542-3.

TABLE 1
B. subtilis strains used

| Strain | Genotype | Source or method |
|---|---|---|
| SMY | wild-type | P. Schaeffer |
| IHA01 | lacA::spc | Härtl, 2001 #145 |
| BB2251 | ΔpdxZ::spc | SMY x pBBI 184 |
| BB2253 | ΔpdxS | SMY x pBB1218 |
| BB2254 | ΔpdxST | SMY x pBB1190 |
| BB2256 | ΔpdxT::ble | SMY x pBB1217 |
| BB2260 | ΔpdxT::ble ΔpdxZ::spc | BB2256 x DNA (BB2251) |
| BB2263 | lacA::spc | SMY x DNA (IHA01) |
| BB2267 | ΔpdxS ΔpdxZ::spc | BB2253 x DNA (BB2251) |
| BB2275 | pdxS::pBB1246[Φ(pdx’T- | SMY x pBB1246 |
| | lacZ cat)] | |
| BB2277 | ΔamyE::Φ(pdxST’-lacZ erm) | SMY x pBB1254 |
| BB2278 | pdxST::Φ(pdxST’-lacZ erm) | SMY x pBB1254 |
| BB2279 | ΔamyE:: Φ(pdxS’-lacZ erm) | SMY x pBB1259 |
| BB2280 | pdxS::pBB1259[Φ(pdxS’- | SMY x pBB1259 |
| | lacZ erm)] | |
| BB2281 | lacA::(Pxyl-pdxS erm) | BB2263 x pBB1249 |
| BB2282 | ΔpxS lacA::(Pxyl-pdxS erm) | BB2253 x DNA (BB2281) |
| BB2283 | dacA::pBB1199(‘dacA) | SMY x pBB1199 |
| BB2285 | ΔamyE::Φ(pdx’ST’-lacZ erm) | SMY x pBB1262 |
| BB2290 | ΔpdxT::ble ΔamyE::Φ(pdxS’- | BB2256 x DNA (BB2279) |
| | lacZ erm) | |
| BB2294 | pdxS::pBB1263[Φ(pdxST’- | SMY x pBB1263 |
| | lacZ erm)] | |
| BB2295 | ΔpdxS ΔpdxZ::spc lacA::(Pxyl- | SMY x pBB1263 |
| | pdxS erm) | |

TABLE 2
Identification of genes of PLP synthesis
in complete bacterial genomes

| Gram-negative bacteria | PdxAJ | PdxST | PdxK/Y | PdxZ | PdxH |
|---|---|---|---|---|---|
| Actinobacillus | — | — | + | − | + |
| actinomycetemcomitans | | | | | |
| Agrobacterium tumefaciens | — | — | + | + | + |
| Anabaena sp. | A₂J^{‡} | — | − | + | + |
| Aquifex aeolicus | AJ | — | − | + | − |
| Bacteroides fragilis | AJ | — | + | + | + |
| Bacteroides thetaiotamicron | AJ | — | +^{#} | + | + |
| Bordetella bronchiseptica | A₂J^{‡} | — | + | + | + |
| Bordetella parapertussis | A₂J^{‡} | — | + | + | + |
| Bordetella pertussis | A₂J^{‡} | — | + | + | + |
| Bradyrhizobium japonicum | A₃J^{‡} | — | + | + | + |
| Brucella melitensis | AJ | — | + | + | + |
| Brucella suis | AJ | — | + | + | + |
| Burkholderia cepacia | A₂J^{‡} | — | + | + | + |
| Burkholderia pseudomallei | AJ | — | + | + | + |
| Campylobacter jejuni | AJ | — | − | + | − |
| Caulobacter crescentus | AJ | — | + | + | + |
| Chlorobium tepidum | AJ | — | − | + | − |
| Coxiella burnetii | AJ | — | − | + | + |
| Deinococcus radiodurans | — | ST | + | + | + |
| Desulfovibric vulgaris | AJ | — | +^{#} | + | − |
| Erwinia carotovora | AJ | — | + | + | + |
| Escherichia coli | AJ | — | + | + | + |
| Fusobacterium nucleatum | A | S* | − | + | − |
| Haemophilus ducreyi | — | ST* | − | + | + |
| Haemophilus influenzae | — | ST | + | + | + |
| Helicobacter pylori | AJ | — | − | + | − |
| Leptospira interrogans | AJ | — | − | + | + |
| Mesorhizobium loti | AJ | — | + | + | + |
| Methylococcus capsulatus | AJ | — | − | + | − |
| Neisseria gonorrhoeae | AJ | — | − | + | + |
| Neisseria meningitidis | AJ | — | − | + | + |
| Nitrosomonas europaea | AJ | — | − | + | − |
| Pasteurella multocida | A | ST* | + | + | − |
| Porphyromonas gingivalis | AJ | — | − | + | + |
| Prochlorococcus marinus | AJ | — | — | + | + |
| Pseudomonas aeruginosa | A₂J^{‡} | — | + | + | + |
| Pseudomonas putida | AJ | − | + | + | + |
| Pseudomonas syringae | AJ | — | + | + | + |
| Ralstonia solanacearum | AJ | — | − | + | + |
| Rhodopseudomonas palustris | AJ | — | + | + | + |
| Salmonella typhi | A₂J^{‡} | — | +* | + | + |
| Salmonella typhimurium | A₂J^{‡} | — | +* | + | + |
| Shewanella oneidensis | AJ | — | − | + | + |
| Shigella flexneri | AJ | — | + | + | + |
| Sinorhizobium meliloti | A₂J^{‡} | — | + | + | + |
| Synechocystis sp. | AJ | — | − | − | + |
| Synechococcus sp. | AJ | — | − | + | + |
| Thermosynechococcus | AJ | — | − | − | + |
| elongates | | | | | |
| Thermotoga maritima | — | ST | − | + | − |
| Vibria cholerae | AJ | — | − | + | + |
| Vibrio parahaemolyticus | AJ | — | + | + | + |
| Vibrio vulnificus | AJ | — | + | + | + |
| Wigglesworthia brevipalpis | AJ | — | − | + | + |
| Wolbachia | J | — | − | − | + |
| Xanthomonas axonopodis | AJ | — | + | + | + |
| Xanthomonas campestris | AJ | — | + | + | + |
| Xylella fastidiosa | AJ | — | − | + | + |
| Yersinia enterocolitica | AJ | — | + | + | + |
| Yersinia pestis | AJ | — | + | + | + |
| Gram-positive bacteria | | | | | |
| Bacillus anthracis | — | ST | − | + | − |
| Bacillus cereus | — | ST | − | + | − |
| Bacillus halodurans | A | ST | − | + | − |
| Bacillus subtilis | — | ST | − | + | − |
| Bifidobacterium longum | — | ST | +^{#} | + | − |
| Clostridium acetobutylicum | — | ST | +^{#} | + | − |
| Clostridium botulinum | A | S | +^{#} | + | − |
| Clostridium perfringens | — | — | +^{#} | + | − |
| Clostridium tetani | — | — | +^{#} | + | − |
| Corynebacterium diphtheriae | — | ST* | + | + | − |
| Corynebacterium efficiens | — | S | + | + | − |
| Corynebacterium glutamicum | — | ST | + | + | − |
| Enterococcus faecalis | — | — | − | + | − |
| Lactobacillus plantarum | — | — | +^{#} | + | − |
| Lactococcus lactis | — | — | − | + | − |
| Listeria innocua | — | ST* | − | + | − |
| Listeria monocytogenes | — | ST* | − | + | − |
| Mycobacterium avium | — | ST | − | + | + |
| Mycobacterium bovis | — | ST | − | + | + |
| Mycobacterium leprae | — | ST | − | + | + |
| Mycobacterium tuberculosis | — | ST | − | + | + |
| Oceanobacillus iheyensis | A | ST* | − | + | − |
| Staphylococcus aureus | — | ST* | − | + | − |
| Staphylococcus epidermidis | — | ST* | − | + | − |
| Streptococcus agalactiae | — | — | − | + | − |
| Streptococcus mutans | — | — | +*^{#} | + | − |
| Streptococcus pneumoniae | — | ST | − | + | − |
| Streptococcus pyogenes | — | — | +*^{#} | + | − |
| Streptomyces avermitilis | — | ST | − | + | + |
| Streptomyces coelicolor | — | ST | − | + | + |
| Thermoanaerobacter | — | ST | − | + | − |
| tengcongensis | | | | | |
| Tropheryma whipplei | — | ST | − | − | − |

^{‡}A₂ or A₃ indicates the presence of multiple pdxA-like genes in a genome.
^{#}Indicates a separate subfamily of PdxK-like proteins.
*Indicates an adjacent location of a putative PLP-dependent transcription regulator [Belitsky, 2002].
No genes of the de novo PLP pathway were detected for most small-genome bacteria. Published genome sequence data were searched by using the NCBI Blast server at http://www.ncbi.nlm.nih.gov/BLAST. Preliminary sequence data were searched at http://www.ncbi.nlm.nih.gov/cgi-bin/Entrez/genom_table_cgi, http://tigrblast.tigr.org/ufmg/, and http://www.sanger.ac.uk/DataSearch/.

TABLE 3
Identification of bacterial species with genomes
having pdxS- and pdxT-like genes

| Gram-positive bacteria | Other bacteria |
|---|---|
| Bacillus anthracis | Actinobacillus pleuropneumoniae |
| Bacillus cereus | Chloroflexus aurantiacus |
| Bacillus circulans | Dehalococcoides ethenogenes |
| Bacillus halodurans | Deinococcus radiodurans |
| Bacillus subtilis | Fibrobacter succinogenes |
| Bifidobacterium longum | Francisella tularensis |
| Carboxydothermus | Fusobacterium nucleatum^{a} |
| hydrogenoformans | |
| Clostridium acetobutylicum | Haemophilus ducreyi |
| Clostridium botulinum^{a} | Haemophilus influenzae |
| Corynebacterium diphtheriae | Pasteurella multocida |
| Corynebacterium efficiens^{a} | Prevotella intermedia |
| Corynebacterium glutamicum | Thermotoga maritima |
| Geobacillus stearothermophilus | Treponema denticola^{a} |
| Listeria innocua | |
| Listeria monocytogenes | |
| Mycobacterium avium | |
| Mycobacterium bovis | |
| Mycobacterium leprae | |
| Mycobacterium tuberculosis | |
| Mycobacterium smegmatis | |
| Oceanobacillus iheyensis | |
| Oenococcus oeni | |
| Ruminococcus albus | |
| Staphylococcus aureus | |
| Staphylococcus epidermidis | |
| Streptococcus pneumoniae | |
| Streptococcus mitis | |
| Streptomyces avermitilis | |
| Streptomyces coelicolor | |
| Thermoanaerobacter | |
| tengcongensis | |
| Thermobifida fusca | |
| Tropheryma whipplei | |

^{a}Indicates the presence of pdxS-like gene only.

TABLE 4
Identification of bacterial species with genomes
having pdxZ-like genes and lacking pdxK/Y-like genes

| Gram-positive bacteria | | Other bacteria | |
|---|---|---|---|
| Bacillus anthracis | +2 | Anabaena sp. | + |
| Bacillus cereus | +2 | Aquifex aeolicus | + |
| Bacillus halodurans | + | Bacteroides thetaiotamicron | +^{#} |
| Bacillus subtilis | +2 | Borrelia burgdorferi | +^{#} |
| Bifidobacterium longum | +^{#} | Campylobacter jejuni | + |
| Clostridium acetobutylicum | +^{#} | Chlorobium tepidum | + |
| Clostridium botulinum | +^{#} | Colwellia psychrerythraea | |
| Clostridium dfficilea | +^{#} | Coxiella burnetii | + |
| Clostridium perfringens | +^{#} | Desulfovibrio vulgaris | +^{#} |
| Clostridium tetani | | Fusobacterium nucleatum | + |
| Enterococcusfaecalis | +3 | Haemophilus ducreyi | ? |
| Enterococcus faecium^{a} | + | Helicobacter pylori | + |
| Lactobacillus plantarum | +^{#} | Leptospira interrogans | + |
| Lactococcus lactis | +2 | Methylococcus capsulatus | ? |
| Listeria innocua | +2 | Neisseria gonorrhoeae | ? |
| Listeria monocytogenes | +2 | Neisseria meningitidis | + |
| Mycobactenium avium | ? | Nitrosomonas europaea | + |
| Mycobactenium bovis | ? | Porphyromonas gingivalis | ? |
| Mycobacterium leprae | + | Prochlorococcus marinus | + |
| Mycobacterium tuberculosis | + | Ralstonia solanacearum | + |
| Oceanobacillus iheyensis | +2 | Shewanella oneidensis | + |
| Staphylococcus aureus | +2 | Synechocystis sp. | ? |
| Staphylococcus epidermidis | +2 | Synechococcus sp. | + |
| Streptococcus agalactiae | +2 | Thermosynechococcus | |
| Streptococcus mutans | +^{#} | elongates | |
| Streptococcus pneumoniae | +2 | Thermotoga maritima | ? |
| Streptococcus pyogenes | +^{#} | Treponema pallidum | + |
| Streptomyces avermitilis | + | Vibrio cholerae | + |
| Streptomyces coelicolor | + | Wigglesworthia brevipalpis | + |
| Thermoanaerobacter | | Wolbachia | ? |
| tengcongensis | + | Xylellafastidiosa | + |
| Tropheryma whipplei | ? | | |

^{a}Indicates an incompletely sequenced genome.
^{#}Indicates a separate subfamily of PdxZ- or PdxK-like proteins.

TABLE 5
Expression of fusion pdxS-lacZ in cells grown in minimal medium

| Swain | Relevant | NH₄Cl, | PL, | Growth | | |
|---|---|---|---|---|---|---|
| activity | genotype | mM | μM | rate | | β-Galactosidase |
| BB2279 | wild-type | 37 | — | 60 | min | 136.0 |
| BB2279 | wild-type | 10 | — | 60 | min | 205.0 |
| BB2279 | wild-type | 10 | 100 | 60 | min | 190.4 |
| BB2279 | wild-type | 120 | — | 60 | min | 133.6 |
| BB2290 | pdxT | 10 | — | 4-6 | hours | 46.5 |
| BB2290 | pdxT | 10 | 100 | 60 | min | 142.4 |
| BB2290 | pdxT | 120 | — | 70 | min | 129.6 |

Cells of each swain were grown to exponential phase in glucose-minimal medium supplemented with NH₄Cl and PL as indicated, and were assayed for β-galactosidase activity.

1. A method of making an avirulent strain of a pathogenic bacterial species, comprising constructing a mutant cell of the species having a non-reverting mutation in a pdx gene encoding an enzyme involved in pyridoxal 5′-phosphate synthesis.

2. The method according to claim 1, wherein the mutation comprises a deletion.

3. The method according to either of claims 1 and 2, wherein the mutation comprises an insertion.

4. The method according to claim 1, wherein the mutation in the pdx gene is at least one mutation in a gene selected from the group pdxS, pdxT, and pdxZ.

5. The method according to claim 1, wherein the mutant cell further comprises apdx gene which is conditionally expressible.

6. The method according to claim 1, wherein the mutant cell has a pyridoxal growth requirement.

7. The method according to claim 6, wherein the growth requirement is not substituted by pyridoxine.

8. The method according to claim 1, wherein growth of the mutant cell is substantially diminished in an infected subject compared to that of a cell of the pathogenic species.

9. The method according to claim 1, wherein the bacterial species is from a genus selected from an Actinobacillus, a Bacillus, a Campylobacter, a Clostridium, a Coxiella, a Corynebacterium, an Ehrlichia, an Enterococcus, a Francisella, a Fusobacterium, a Haemophilus, a Helicobacter, a Legionella, a Leptospira, a Listeria, a Mannheimia, a Mycobacterium, a Neisseria, a Neorickettsia, a Pasteurella, a Porphyromonas, a Prevotella, a Ralstonia, a Staphylococcus, and a Streptococcus, a Treponema, a Tropheryma, a Vibrio, a Wigglesworthia, and a Xylella.

10. The method according to claim 9, wherein the bacterial species is selected from the group of Actinobacillus pleuropneumoniae, Bacillus anthracis, B. cereus, Campylobacter jejeuni, Clostridium botulinum, Coxiella burnetti, Corynebacterium diphtheria, Enterococcus faecalis, Enterococcus faecium, Ehrlichia chaffeensis, Fusobacterium nucleatum, Francisella tularensis, Haemophilus ducreyi, Haemophilus influenzae, Helicobacter pylori, Legionella pneumophila, Leptospira interrogans, Listeria monocytogenes, Mannheimia haemolytica, Mycobacterium avium, M bovis, M leprae, M tuberculosis, Neisseria meningitidis, Pasteurella multocida, Prevotella intermedia, Ralstonia solanacearum, Staphylococcus aureus, S. epidermidis, S haemolyticus, Streptococcus agalactiae, S. mitis, S. pneumoniae, S sobrinus, S. uberus, Treponema denticola, T. pallidum, Tropheryma whipplei, Vibrio cholerae, Wigglesworthia brevipalpis, and Xylella fastidiosa.

11. An avirulent strain produced according to the method of claim 1.

12. An avirulent strain produced according to the method of claim 10, wherein the Clostridium is C botulinum, the Bacillus is B. anthracis or B. cereus, the Campylobacter is C jejuni, the Corynebacterium is C. diphtheriae, the Enterococcus is E. faecalis or E. faecium, the Francisella is F tularensis, the Fusobacterium is Fusobacterium nucleatum, the Haemophilus is H ducreyi or H. influenzae, the Leptospira is L. interrogans, the Listeria is L. monocytogenes, the Mycobacterium is selected from the group consisting of M avium, M bovis, M leprae, and M tuberculosis, the Staphylococcus is selected from the group consisting of S. aureus, S. epidermidis, S. haemolyticus, and the Streptococcus is selected from the group consisting of S. agalactiae, S. mitis, S. pneumoniae, S. sobrinus, S. uberus.

13. A vaccine composition prepared according to any of the methods of claims 1-10.

14. The vaccine composition according to claim 13 in an effective dose.

15. The vaccine composition according to claim 13 further comprising an adjuvant.

16. The vaccine composition according to claim 13 further comprising a pharmaceutically acceptable carrier.

17. A kit comprising a container and a vaccine according to any of claims 13-16.

18. A kit according to claim 17, further comprising instructions for use.

19. A method of identifying from among a plurality of chemical compounds a potential antimicrobial agent that is inhibitory for synthesis of pyridoxal 5′-phosphate (PLP), the method comprising:
contacting a first sample of cells of a bacterial test strain with at least one of the plurality of compounds, wherein the strain has a pathway of de novo PLP synthesis; and
comparing growth of the first sample with that of a second sample not so contacted and otherwise identical, and with a third sample similarly contacted and in the presence of excess vitamin B₆, such that inhibition of growth of cells in the first sample in comparison to growth of cells in the second and third samples is an indication that the compound is an agent inhibitory fo PLP synthesis.

20. The method of claim 19, wherein the de novo pathway of synthesis in the test strain is a PdxST pathway.

21. The method according to claims 19, wherein the test strain is selected from the group consisting of: Bacillus subtilis, Geobacillus stearothermophilus, and Listeria monocytogenes.

22. The method according to claim 20, further comprising contacting a sample of cells of a control strain having a non-PdxST de novo PLP pathway for PLP synthesis with the at least one compound, wherein lack of inhibition of growth of cells of the second strain is a further indication that the compound is an inhibitor of the PdxST pathway.

23. The method according to claim 22, wherein the non-PdxST de novo pathway is a PdxAJ pathway.

24. The method according to claim 22, wherein the control strain is an E. coli.

25. The method according to claims 19-24, wherein the test strain is selected from the group consisting of: Actinobacillus pleuropneumoniae, Bacillus anthracis, B. cereus, Campylobacterjejeuni, Clostridium botulinum, Corynebacterium diphtheria, Coxiella burnetti, Ehrlichia chaffeensis, Enterococcus faecalis, E. faecium, Fusobacterium nucleatum, Francisella tularensis, Haemophilus ducreyi, Haemophilus influenzae, Helicobacter pylori, Legionella pneumophila, Leptospira interrogans, Listeria monocytogenes, Mannheimia haemolytica, Mycobacterium avium, Mycobacterium bovis, Mycobacterium leprae, M tuberculosis, Neisseria gonorrhoeae, N. meningitidis, Neorickettsia sennettsu, Pasteurella multocida, Prevotella intermedia, Porphyromonas gingivalis, Ralstonia solanacearum, Staphylococcus aureus, S. epidermidis, S. haemolyticus, Streptococcus agalactiae, S. mitis, S. pneumoniae, S. sobrinus, S. uberus, Treponema denticola, T pallidum, Tropheryma whipplei, Vibrio cholerae, Wigglesworthia brevipalpis, and Xylella fastidiosa.

26. A method of identifying from among a plurality of chemical compounds a potential antimicrobial agent that is inhibitory for synthesis of pyridoxal 5′-phosphate (PLP), the method comprising:
contacting a first sample of cells of a bacterial strain grown in the presence of vitamin B₆ with at least one of the plurality of compounds, wherein the strain has a PLP salvage pathway having a PdxZ pyridoxal kinase and mutationally lacking a de novo PLP synthesis pathway; and
comparing growth of the first sample with a second sample of cells not contacted with the compound and otherwise identical and with a third sample of the bacterial strain otherwise identical and in the presence of an effective amount of a B₆ vitamer, wherein inhibition of growth of the first sample compared to the second sample and the third sample indicates that the compound is an inhibitor of synthesis of PLP.

27. The method according to claim 26, wherein the effective amount for growth of the third sample of cells is at least 1 mM of the vitamin B₆ vitamer.

28. The method according to claim 26, wherein the effective amount for growth of the third sample of cells is at least at least 2 mM of the vitamin B₆ vitamer.

29. The method according to claim 26, wherein the bacterial species is from a genus selected from an Actinobacillus, a Bacillus, a Campylobacter, a Clostridium, a Coxiella, a Corynebacterium, an Ehrlichia, an Enterococcus, a Francisella, a Fusobacterium, a Haemophilus, a Helicobacter, a Legionella, a Leptospira, a Listeria, a Mannheimia, a Mycobacterium, a Neisseria, a Neorickettsia, a Pasteurella, a Porphyromonas, a Prevotella, a Ralstonia, a Staphylococcus, a Streptococcus, a Treponema, a Tropheryma, a Vibrio, a Wigglesworthia, and a Xylella.

30. A method of identifying from among a plurality of chemical compounds a potential antimicrobial agent that is inhibitory for synthesis of pyridoxal 5′-phosphate (PLP), the method comprising:
contacting a first sample of a bacterial PdxS-PdxT enzyme complex with at least one of the plurality of compounds; and
comparing enzymatic activity of the first sample with that of a second sample not so contacted and otherwise identical, wherein inhibition of activity in the first sample in comparison to activity in the second samples is an indication that the compound is an inhibitor of PLP synthesis.

31. The method according to claim 30, further comprising a third and a fourth sample having an enzyme that is not PdxS-PdxT, the third sample being contacted with the compound and the fourth sample not so contacted and otherwise identical to the third, wherein absence of inhibition of the third sample in comparison to the fourth sample is a further indication that the compound is a specific inhibitor of PdxS-PdxT.

32. The method according to claim 31, wherein the enzyme in the third and fourth samples is selected from the group consisting of β-galactosidase, alkaline phosphatase, α-amylase, and horse radish peroxidase.

33. The method according to claim 30, wherein comparing enzymatic activity is measuring a glutaminase activity.

34. The method according to claim 30, wherein the bacterial PdxS-PdxT enzyme is a bacterial strain selected from the group of a B. subtilis, a G. stearothermophilus, and a Listeria monocytogenes strain.

35. The method according to claim 30, wherein the bacterial strain is from a bacterioal strain selected from the group of an Actinobacillus, a Bacillus, a Campylobacter, a Clostridium, a Coxiella, a Corynebacterium, an Ehrlichia, an Enterococcus, a Francisella, a Fusobacterium, a Haemophilus, a Helicobacter, a Legionella, a Leptospira, a Listeria, a Mannheimia, a Mycobacterium, a Neisseria, a Neorickettsia, a Pasteurella, a Porphyromonas, a Prevotella, a Ralstonia, a Staphylococcus, a Streptococcus, a Treponema, a Tropheryma, a Vibrio, a Wigglesworthia, and a Xylella.

36. The method according to claim 33, wherein the enzyme is isolated.

37. The method according to claim 36, wherein the enzyme further comprises a modification.

38. The method according to claim 36, wherein at least one of PdxS or PdxT comprises additional histidine residues.
